(19) United States
(12) Patent Application Publication
(10) Pub. No.: US 2024/0016983 A1
(43) Pub. Date: Jan. 18, 2024

(54) Hybrid Micro Molding-Fiber Deposition Substrate Processing for Cell Biology Manipulation and Local Anisotropy

(71) Applicants: University of Pittsburgh - Of the Commonwealth System of Higher Education, Pittsburgh, PA (US); Ri.MED FOUNDATION, Palermo (IT)

(72) Inventors: William R. Wagner, Gibsonia, PA (US); Pietro Terranova, Palermo (IT); Arianna Adamo, Messina (IT); Antonio D'Amore, Pittsburgh, PA (US)

(21) Appl. No.: 18/248,925

(22) PCT Filed: Oct. 13, 2021
(86) PCT No.: PCT/US2021/054767

§ 371 Date: Apr. 13, 2023

Related U.S. Application Data
(60) Provisional application No. 63/091,462, filed on Oct. 14, 2020.

(51) Int. Cl. A61L 27/48; D01D 5/00; B33Y 10/00; B33Y 80/00; B33Y 30/00; A61L 27/50; A61L 27/38

(52) U.S. Cl. CPC A61L 27/48; D01D 5/003; D01D 5/0076; B33Y 10/00; B33Y 80/00; B33Y 30/00; A61L 27/507; A61L 27/3826; D10B 2509/00; D10B 2331/30; A61L 2430/20; A61L 2430/40

(57) ABSTRACT

Methods, systems, and devices are provided herein for preparing fiber matrices with differing degrees of anisotropy within a single matrix and controllable physical parameters, such as porosity.

### CROSS REFERENCE TO RELATED APPLICATIONS

This application claims the benefit of U.S. Provisional Application No. 63/091,462, filed Oct. 14, 2020, which is incorporated herein by reference in its entirety.

Provided herein are methods of manufacturing complex articles for use in biological systems. Also provided are electrodeposition devices and systems for implementation of the manufacturing method.

Biomaterial surface modifications to alter cell interaction with the substrate is a rapidly-growing field of research. Changes in material topology and texture play a crucial role in cell behavior. In particular, geometric modification of the surface pattern at the meso-scale (e.g., 10-250 μm) can affect key elements in cell biology including: cell proliferation, cell migration, collagen elaboration, and phenotype expression.

Biomimicry in scaffold design has increasingly become a popular paradigm in tissue engineering. Reproducing native tissue extracellular matrix (ECM) architectures is considered to be one of the most straightforward approaches to restore the natural three-dimensional niche able to facilitate cell adhesion, proliferation, differentiation, and neo tissue genesis.

An ideal modified substrate interface should provide adequate mechanical support, inhibit non-specific tissue growth, show similarity with the native tissue structural organization, guarantee exchange of nutrients and cell waste, and allow cells to move and colonize the scaffold volume. In turn, these factors are essential for the development and elaboration of ex-novo tissue. Due to the complexity in processing biomaterials with such surface characteristics at the mesoscopic scale cell—medical device interactions often remain sub-optimal.

For example, Endothelial Cells (ECs) normally spread poorly on flat substrates (e.g., DACRON®, TEFLON®, or steel) with over physiological stiffness (MPa-GPa range) that are commonly used for blood-contacting medical devices. The endothelium is a continuous monolayer of ECs that covers the entire cardiovascular system interior surface. ECs are anchored to a thin sheet of specialized ECs, the Basal Membrane (BM). The native endothelial BM is an example of an ideal substrate optimized to promote EC growth, adhesion, and structural organization. Its topography is a meshwork with characteristic lengths of its fibers and pores at the mesoscopic scale. Endothelium and BM together are responsible for regulating important physiological mechanisms such as hemostasis and thrombosis. Given the crucial role of substrate topography, biomaterial processing methods are increasingly focusing on better technologies to duplicate native tissue surface patterns at the different scale lengths.

Blood-contacting medical devices, such as catheters for dialysis, stents, prosthetic heart valves, oxygenators, and vascular grafts are widely used to treat patients in different clinical contexts. The interaction between blood and the medical device's artificial surface triggers coagulation and thrombus formation. The activation of the coagulation cascade and the consequent inflammation induces the thrombotic state in patients. This condition, which remains a physiological response to a foreign body and part of the wound healing process, may result in the context of host—medical device interaction in peripheral ischemia, pulmonary embolism, or heart attack. To prevent this condition, patients are treated with lifelong therapy, using anticoagulants and/or platelet inhibitors. However, such systemic treatments are associated with risk of major bleeding, stroke and hemorrhage. While several material surface chemical or biological functionalization strategies have been introduced to reduce the thrombogenicity of medical devices, the most natural approach remains to cover the material surface with host derived ECs or to manipulate the surface interface to elicit such effect. Even not considering the vast spectrum of potential surface chemistry modifications, this notion requires overcoming numerous challenges related to the biophysics of the substrate. This includes: identifying the most effective geometry and substrate mechanics, and, therefore, developing processing technologies that allow for adequate control of meso scale topology, and at the same time, permitting process scalability to the organ level size (cm).

### SUMMARY

A method of making a polymer matrix structure with controlled fiber alignment is provided. The method comprises, electrodepositing polymer fibers from an electrodeposition nozzle onto a surface of a target comprising a non-homogenous (that is, having a different pattern, orientation, size, etc. on two or more different portions or sections of the surface of the target) pattern of alternating ridges and valleys extending generally perpendicularly to a fiber alignment direction to produce an aligned electrical field for depositing the polymer fibers in the fiber alignment direction, optionally wherein the periodicity, that is, the distance between centers of adjacent ridges, is 500 μm or less, 250 μm or less, 150 μm or less, 100 μm or less, or 50 μm or less and/or the valleys having a depth of 15 μm or less, 14 μm or less, 13 μm or less, 12 μm or less, 11 μm or less, 10 μm or less, 9 μm or less, 8 μm or less, 7 μm or less, or 6 μm or less, such as less than 50 μm, ranging from 15 μm to 150 μm, or ranging from 15 μm to less than 50 μm.

A method of making a polymer matrix structure with controlled fiber alignment is provided, comprising, electrodepositing polymer fibers from an electrodeposition nozzle onto a surface of a non-planar target wherein the electrodeposition nozzle is moved relative to the target, optionally using a robotic device.

A method of making a polymer matrix structure with controlled fiber alignment is provided. The method comprises, depositing polymer fibers from a deposition nozzle onto a surface of a siloxane target comprising a siloxane composition, the target comprising a repeated three-dimensional pattern (e.g., tessellation) of raised shapes with a periodicity (e.g., the sum of frequency and width as shown in FIG. 4A-4E, or the distance between equivalent points on each adjacent raised shape) of less than 500 μm, 250 μm or less, 150 μm or less, 100 μm or less, or 50 μm or less and/or the valleys having a depth of 15 μm or less, 14 μm or less, 13 μm or less, 12 μm or less, 11 μm or less, 10 μm or less, 9 μm or less, 8 μm or less, 7 μm or less, or 6 μm or less, such as less than 50 μm, ranging from 15 μm to 150 μm, or ranging from 15 μm to less than 50 μm.

A system for the production of variably anisotropic fiber matrices is provided, comprising:
an electrodeposition target attached to an electrical voltage source comprising: an electrodeposition surface comprising a non-homogenous (that is, having a different pattern, orientation, size, etc. over the surface of the target) pattern of alternating ridges and valleys extending generally perpendicularly to a fiber alignment direction to produce an aligned electrical field for depositing the polymer fibers in the fiber alignment direction, optionally wherein the periodicity, that is, the distance between centers of adjacent ridges, is 500 μm or less, 250 μm or less, 150 μm or less, 100 μm or less, or 50 μm or less and/or the valleys having a depth of 15 μm or less, 14 μm or less, 13 μm or less, 12 μm or less, 11 μm or less, 10 μm or less, 9 μm or less, 8 μm or less, 7 μm or less, or 6 μm or less, such as less than 50 μm, ranging from 15 μm to 150 μm, or ranging from 15 μm to less than 50 μm; or a siloxane composition having an electrodeposition surface comprising a repeated three-dimensional pattern (e.g., tessellation) of raised shapes with a periodicity of less than 500 μm, 250 μm or less, 150 μm or less, 100 μm or less, or 50 μm or less and/or the valleys having a depth of 15 μm or less, 14 μm or less, 13 μm or less, 12 μm or less, 11 μm or less, 10 μm or less, 9 μm or less, 8 μm or less, 7 μm or less, or 6 μm or less, such as less than 50 μm, ranging from 15 μm to 150 μm, or ranging from 15 μm to less than 50 μm;
an electrodeposition nozzle attached to an electrical voltage source; and
a polymer solution reservoir and pump configured to supply a polymer solution to the electrodeposition nozzle.

A method of making a vascular graft or heart valve graft is provided, comprising: seeding a polymer matrix structure prepared according to any method described herein, shaped as a vascular graft or heart valve, with endothelial cells, vascular smooth muscle cells, or precursors of endothelial cells or vascular smooth muscle cells;
if precursors of endothelial cells or vascular smooth muscle cells are seeded, culturing the polymer matrix structure under conditions for differentiating the precursor cells into endothelial cells or vascular smooth muscle cells; and culturing the cells to expand the number of cells on the polymer matrix structure.

A method of making a graft for tissue replacement or repair in a patient is provided, comprising:
seeding a polymer matrix structure prepared according to any method described herein, shaped as a graft for the tissue to be repaired or replaced, with cells or precursors of the cells;
if precursors of the cells are seeded, culturing the polymer matrix structure under conditions for differentiating the precursor cells into cells of the tissue to be repaired or replaced; and culturing the cells to expand the number of cells on the polymer matrix structure.

A method of repairing or replacing tissue is provided, such as vascular or heart valve tissue, in a patient, comprising implanting in the patient, e.g., at a site for repair or replacement of the tissue in the patient, a polymer matrix structure or graft prepared according to any method described herein.

A system for use in preparing a three-dimensional article comprising variably anisotropic fiber matrices is provided, comprising:
an electrodeposition target attached to an electrical voltage source comprising a non-planar electrodeposition surface;
an electrodeposition nozzle attached to an electrical voltage source and configured to move relative to the electrodeposition target;
a polymer solution reservoir and pump configured to supply a polymer solution to the electrodeposition nozzle; and
a controller for executing computer-implemented instructions for controlling position and movement of the electrodeposition nozzle over and relative to the target surface, and optionally the relative charge of the electrodeposition target and the electrodeposition nozzle and/or flow of polymer solution through the electrodeposition nozzle.

The following numbered clauses describe various aspects and/or embodiments of the present invention.

Clause 1. A method of making a polymer matrix structure with controlled fiber alignment, comprising, electrodepositing polymer fibers from an electrodeposition nozzle onto a surface of a target comprising a non-homogenous pattern of alternating ridges and valleys extending generally perpendicularly to a fiber alignment direction to produce an aligned electrical field for depositing the polymer fibers in the fiber alignment direction, optionally wherein the periodicity, that is, the distance between centers of adjacent ridges, is 500 μm or less, 250 μm or less, 150 μm or less, 100 μm or less, or 50 μm or less and/or the valleys having a depth of 15 μm or less, 14 μm or less, 13 μm or less, 12 μm or less, 11 μm or less, 10 μm or less, 9 μm or less, 8 μm or less, 7 μm or less, or 6 μm or less, such as less than 50 μm, ranging from 15 μm to 150 μm, or ranging from 15 μm to less than 50 μm.

Clause 2. The method of clause 1, wherein the target and/or surface of the target is conductive.

Clause 3. The method of any one of clause 1 or 2, wherein the target comprises a siloxane composition comprising the plurality of alternating ridges and valleys.

Clause 4. The method of clause 3, wherein the siloxane composition comprises polydimethylsiloxane (PDMS).

Clause 5. The method of clause 3 or 4, wherein the siloxane composition comprises a conductor.

Clause 6. The method of clause 5, wherein the conductor comprises conductive particles, such as metal nanoparticles.

Clause 7. The method of clause 5, wherein the siloxane is doped with a conductive material.

Clause 8. The method of clause 5, wherein the conductor comprises polyaniline, such as a polyaniline particle.

Clause 9. The method of clause 5, wherein the conductor comprises a conductive carbon allotrope, such as graphite, amorphous carbon, carbon black, graphene, or carbon nanotubes.

Clause 10. The method of any one of clauses 1-9, wherein the target further comprises a conductive layer deposited over at least a portion of the surface of the target.

Clause 11. The method of clause 1 or 2, wherein the surface of the target comprising the pattern of alternating ridges and valleys is metallic, such as wherein the target is a metal target.

Clause 12. The method of any one of clauses 1-11, wherein at least a pair of adjacent ridges have polygonal cross-sections.

Clause 13. The method of any one of clauses 1-12, wherein one or both of the target and the electrodeposition nozzle move in a direction relative to each other during the electrodeposition, and at least a portion of the ridges and valleys of the target are not longitudinally extended perpendicular to a direction of relative movement of the target and the electrodeposition nozzle, thereby depositing a fiber in a direction not parallel to the direction of relative movement of the target and the electrodeposition nozzle.

Clause 14. The method of any one of clauses 1-13, wherein the target is a rotating mandrel or the target is static, and the electrodeposition nozzle is moved relative to the target, optionally using a robotic device.

Clause 15. The method of clause 14, wherein the target is static, and the electrodeposition nozzle is moved relative to the target using a robotic device controlled by a computer-implemented process, such as a process implementing a movement pattern for preparation of a structure in a computer-aided design or manufacturing (e.g., CAD/CAM) file.

Clause 16. The method of any one of clauses 1-15, wherein the target comprises a pattern of ridges and valleys configured to produce a fiber matrix in the size and shape of a heart valve leaflet having a base edge (the edge of the heart valve leaflet closest to the heart valve annulus) and an apex (the edge of the heart valve leaflet most distal to the base or the heart valve annulus), and wherein the ridges and valleys are arranged to produce a convex arrangement of fibers with respect to the base edge, optionally recapitulating anisotropy in native heart valves.

Clause 17. A method of making a polymer matrix structure with controlled fiber alignment, comprising, electrodepositing polymer fibers from an electrodeposition nozzle onto a surface of a non-planar target wherein the electrodeposition nozzle is moved relative to the target, optionally using a robotic device.

Clause 18. The method of clause 17, wherein the target is static and the electrodeposition nozzle is moved relative to the target during electrodeposition.

Clause 19. The method of clause 17 or 18, wherein the target has protruding and recessed features, and the electrodeposition nozzle is maintained at a substantially fixed distance from the target during electrodeposition.

Clause 20. The method of any one of clauses 17-19, wherein different amounts of the polymer fibers are electrodeposited over different portions of the target to produce a polymer matrix structure having portions with different thicknesses.

Clause 21. The method of any one of clauses 17-20, the target comprising a non-homogenous (that is, having a different pattern, orientation, size, etc. on two or more different portions or sections of the deposition surface of the target) pattern of alternating ridges and valleys extending generally perpendicularly to a fiber alignment direction to produce an aligned electrical field for depositing the polymer fibers in the fiber alignment direction, optionally wherein the periodicity, that is, the distance between centers of adjacent ridges, is 500 μm or less, 250 μm or less, 150 μm or less, 100 μm or less, or 50 μm or less and/or the valleys having a depth of 15 μm or less, 14 μm or less, 13 μm or less, 12 μm or less, 11 μm or less, 10 μm or less, 9 μm or less, 8 μm or less, 7 μm or less, or 6 μm or less, such as less than 50 μm, ranging from 15 μm to 150 μm, or ranging from 15 μm to less than 50 μm.

Clause 22. The method of any one of clauses 17-20, wherein the target comprises a pattern of ridges and valleys configured to produce a fiber matrix in the size and shape of a heart valve leaflet having a base edge (the edge of the heart valve leaflet closest to the heart valve annulus) and an apex (the edge of the heart valve leaflet most distal to the base or the heart valve annulus), and wherein the ridges and valleys are arranged to produce a convex arrangement of fibers with respect to the base edge, optionally recapitulating anisotropy in native heart valves.

Clause 23. The method of clause 22, wherein the target is shaped to produce a fiber matrix in the shape of a heart valve.

Clause 24. A method of making a polymer matrix structure with controlled fiber alignment, comprising, depositing polymer fibers from a deposition nozzle onto a surface of a siloxane target comprising a siloxane composition, the target comprising a repeated three-dimensional pattern (e.g., tessellation) of raised shapes with a periodicity (e.g., the sum of frequency and width as shown in FIG. 4A-4E, or the distance between equivalent points on each adjacent raised shape) of less than 500 μm, 250 μm or less, 150 μm or less, 100 μm or less, or 50 μm or less and/or the valleys having a depth of 15 μm or less, 14 μm or less, 13 μm or less, 12 μm or less, 11 μm or less, 10 μm or less, 9 μm or less, 8 μm or less, 7 μm or less, or 6 μm or less, such as less than 50 μm, ranging from 15 μm to 150 μm, or ranging from 15 μm to less than 50 μm.

Clause 25. The method of clause 24, wherein the siloxane composition comprises polydimethylsiloxane (PDMS).

Clause 26. The method of clause 24 or 25, wherein the siloxane composition comprises a conductor.

Clause 27. The method of clause 26, wherein the conductor comprises conductive particles, such as metal nanoparticles.

Clause 28. The method of clause 26 wherein the siloxane is doped with a conductor.

Clause 29. The method of clause 26, wherein the conductor comprises polyaniline, such as a polyaniline particle.

Clause 30. The method of clause 26, wherein the conductor comprises a conductive carbon allotrope, such as graphite, amorphous carbon, carbon black, graphene, or carbon nanotubes.

Clause 31. The method of any one of clauses 24-30, wherein the target further comprises a conductive layer deposited over at least a portion of the surface of the target.

Clause 32. The method of any one of clauses 24-31, wherein the shapes of repeated three-dimensional pattern are complex, and/or are not square or rectangular.

Clause 33. The method of any one of clauses 24-32, wherein the polymer fibers are electrodeposited from an electrodeposition nozzle.

Clause 34. The method of any one of clauses 24-33, wherein the target is static, and the deposition nozzle is moved relative to the target using a robotic device.

Clause 35. The method of clause 34, wherein the robotic device is controlled by a computer-implemented process, such as from a computer-aided design or manufacturing (e.g., CAD/CAM) file.

Clause 36. The method of any one of clauses 1-35, wherein the polymer fibers are prepared from a bioerodable polymer.

Clause 37. The method of clause 36, wherein the bioerodable polymer is a polyurethane, a polyester, a polyester-containing copolymer, a polyanhydride, a polyanhydride-containing copolymer, a polyorthoester, a polymer comprising monomers derived from an alpha-hydroxy acid, and a polyorthoester-containing copolymer.

Clause 38. The method of clause 36, wherein the bioerodable polymer of the polymer fibers is one or more polymer selected from the group consisting of: a poly(lactic-co-glycolic) acid (PLGA); a poly(lactic acid) (PLA); a poly(trimethylene carbonate) (PTMC); a poly(caprolactone) (PCL); a poly(glycolic acid) (PGA); a poly(glycolide-co-trimethylenecarbonate) (PGTMC); a polyethylene-glycol (PEG-) containing block copolymer; a polyphosphazene; a poly(ester urethane) urea (PEUU); a poly(ether ester urethane)urea (PEEUU); a poly(ester carbonate)urethane urea (PECUU); a poly(carbonate)urethane urea (PCUU); a polylactide; a poly(lactide-co-glycolide); a poly(I-lactide-co-caprolactone); a polyglycolic acid; a poly(dl-lactide-co-glycolide); a poly(I-lactide-co-dl-lactide); a polyhydroxybutyrate; a polyhydroxyvalerate; a polydioxanone; a polyglactin; a polycaprolactone; a polycarbonate; a polyglyconate; a poly(glycolide-co-trimethylene carbonate); and a poly(glycolide-co-trimethylene carbonate-co-dioxanone).

Clause 39. A system for the production of variably anisotropic fiber matrices, comprising:
an electrodeposition target attached to an electrical voltage source comprising:
   an electrodeposition surface comprising a non-homogenous (that is, having a different pattern, orientation, size, etc. over the surface of the target) pattern of alternating ridges and valleys extending generally perpendicularly to a fiber alignment direction to produce an aligned electrical field for depositing the polymer fibers in the fiber alignment direction, optionally wherein the periodicity, that is, the distance between centers of adjacent ridges, is 500 μm or less, 250 μm or less, 150 μm or less, 100 μm or less, or 50 μm or less and/or the valleys having a depth of 15 μm or less, 14 μm or less, 13 μm or less, 12 μm or less, 11 μm or less, 10 μm or less, 9 μm or less, 8 μm or less, 7 μm or less, or 6 μm or less, such as less than 50 μm, ranging from 15 μm to 150 μm, or ranging from 15 μm to less than 50 μm; or
   a siloxane composition having an electrodeposition surface comprising a repeated three-dimensional pattern (e.g., tessellation) of raised shapes with a periodicity of less than 500 μm, 250 μm or less, 150 μm or less, 100 μm or less, or 50 μm or less and/or the valleys having a depth of 15 μm or less, 14 μm or less, 13 μm or less, 12 μm or less, 11 μm or less, 10 μm or less, 9 μm or less, 8 μm or less, 7 μm or less, or 6 μm or less, such as less than 50 μm, ranging from 15 μm to 150 μm, or ranging from 15 μm to less than 50 μm;
an electrodeposition nozzle attached to an electrical voltage source; and a polymer solution reservoir and pump configured to supply a polymer solution to the electrodeposition nozzle.

Clause 40. A method of making a vascular graft or heart valve graft, comprising:
seeding a polymer matrix structure prepared according to any one of clauses 1-31, shaped as a vascular graft or heart valve, with endothelial cells, vascular smooth muscle cells, or precursors of endothelial cells or vascular smooth muscle cells;
if precursors of endothelial cells or vascular smooth muscle cells are seeded, culturing the polymer matrix structure under conditions for differentiating the precursor cells into endothelial cells or vascular smooth muscle cells; and
culturing the cells to expand the number of cells on the polymer matrix structure.

Clause 41. A method of making a graft for tissue replacement or repair in a patient, comprising:
seeding a polymer matrix structure prepared according to any one of clauses 1-31, shaped as a graft for the tissue to be repaired or replaced, with cells or precursors of the cells;
if precursors of the cells are seeded, culturing the polymer matrix structure under conditions for differentiating the precursor cells into cells of the tissue to be repaired or replaced; and
culturing the cells to expand the number of cells on the polymer matrix structure.

Clause 42. A method of repairing or replacing tissue, such as vascular or heart valve tissue, in a patient, comprising implanting in the patient, e.g., at a site for repair or replacement of the tissue in the patient, a polymer matrix structure or graft prepared according to any one of clauses 1-31, 40, or 41.

Clause 43. A system for use in preparing a three-dimensional article comprising variably anisotropic fiber matrices, comprising:
an electrodeposition target attached to an electrical voltage source comprising a non-planar electrodeposition surface;
an electrodeposition nozzle attached to an electrical voltage source and configured to move relative to the electrodeposition target;
a polymer solution reservoir and pump configured to supply a polymer solution to the electrodeposition nozzle; and
a controller for executing computer-implemented instructions for controlling position and movement of the electrodeposition nozzle over and relative to the target surface, and optionally the relative charge of the electrodeposition target and the electrodeposition nozzle and/or flow of polymer solution through the electrodeposition nozzle.

Clause 44. The system of clause 43, wherein the electrodeposition target is static, and the electrodeposition nozzle is moved relative to the electrodeposition surface during electrodeposition.

Clause 45. The system of clause 43 or 44, wherein the electrodeposition surface comprises a non-homogenous (that is, having a different pattern, orientation, size, etc. over the surface of the target) pattern of alternating ridges and valleys extending generally perpendicularly to a fiber alignment direction to produce an aligned electrical field for depositing the polymer fibers in the fiber alignment direction, optionally wherein the periodicity, that is, the distance between centers of adjacent ridges, is 500 μm or less, 250 μm or less, 150 μm or less, 100 μm or less, or 50 μm or less and/or the valleys having a depth of 15 μm or less, 14 μm or less, 13 μm or less, 12 μm or less, 11 μm or less, 10 μm or less, 9 μm or less, 8 μm or less, 7 μm or less, or 6 μm or less, such as less than 50 μm, ranging from 15 μm to 150 μm, or ranging from 15 μm to less than 50 μm.

Clause 46. The system of clause 43 or 44, wherein the electrodeposition surface comprises a siloxane composition having an electrodeposition surface comprising a repeated three-dimensional pattern (e.g., tessellation) of raised shapes with a periodicity of less than 500 μm, 250 μm or less, 150 μm or less, 100 μm or less, or 50 μm or less and/or the valleys having a depth of 15 μm or less, 14 μm or less, 13 μm or less, 12 μm or less, 11 μm or less, 10 μm or less, 9 μm or less, 8 μm or less, 7 μm or less, or 6 μm or less, such as less than 50 μm, ranging from 15 μm to 150 μm, or ranging from 15 μm to less than 50 μm.

### BRIEF DESCRIPTION OF THE DRAWINGS

FIGS. 1A-1F are images depicting the electrodeposition process. FIG. 1A is a schematic depicting the stereo-photolithography and casting process for PDMS mold production. FIG. 1B is an example of a master mold design with four different micro patterns of different shape and size. FIG. 1C is an image of the master mold obtained after stereo-photolithography process. FIG. 1D is an image of the PDMS micro mold after drying. FIG. 1E is an image of an example of PDMS micro mold for electrospinning on flat static electrode. FIG. 1F is an image of the electrospinning—micro molding process, where micro-fibers are deposited onto a selected PDMS mold placed on a flat conductive collector.

FIGS. 2A-2B are graphs depicting the conic angle versus the applied electrical potential (kV). FIG. 2A is a graph depicting the conic angle versus the applied electrical potential (kV) at a flow rate of 0.4 ml/h. FIG. 2B is a graph depicting the conic angle versus the applied electrical potential (kV) at a flow rate of 0.8 ml/h.

FIGS. 3A-3C are images showing the electrospinning process. FIG. 3A is fluorescence microscopy image of the PDMS mold having square shapes. FIG. 3B is a fluorescence microscopy image of the electrospun 12% PEUU solution on the PDMS mold, creating a micro-patterned fibrous tissue. FIG. 3C is a scanning electron microscopy (SEM) image of the electrospun 12% PEUU solution on the PDMS mold, creating a micro-patterned fibrous tissue.

FIGS. 4A-4E show the combination of Double Component Deposition (DCD) and micro-grooves (μDCD). FIG. 4A is a photograph showing that mandrel tangential velocity dictates anisotropy on DCD mandrel for mitral valve fabrication (FIG. 4B). FIG. 4C is a photograph of a copper rod that was used to investigate the impact of different grooved configurations on scaffold structural and mechanical anisotropy. The three parameters that were studied included: frequency, width, and depth. FIG. 4D is a schematic showing the electrospinning layout with dynamic target where the tangential velocity affects the fiber alignment and the mechanical anisotropy. FIG. 4E is an SEM analysis of Hybrid Micro-molding Electrodeposition Surface (HMES) groove.

FIGS. 5A-5C are images depicting the electrospinning process. FIG. 5A is a photograph showing the macroscopic view of the micro mold after the fiber deposition. FIG. 5B is a Scanning Electron Microscopy (SEM) image of the micro mold after the fiber deposition. Note the polymeric pattern obtained at the meso scale and the micro fibers. FIG. 5C is a SEM image showing the detail from FIG. 5B showing pattern and fibers.

FIGS. 6A and 6B depict cell proliferation on micro-patterned films and scaffolds. FIG. 6A is a SEM analysis of HMES with different patterns prepared essentially as described with respect to FIGS. 1A-1F and 5A-5C. Two different magnifications are shown for each (left-right). Starting from the top: fish scale, herringbone, Honeycomb. The inset image shows fluorescent microscope analysis of electrospun fabrication labeled with a fluorescent probe. FIG. 6B is a graph depicting vascular smooth muscle cell (VSMC) Alamar Blue vitality test at 24 hours. Cells proliferation on micro-pattern with casted solid geometry and hybrid electrospun micro patterned samples were tested after 24 hours (One Way ANOVA*=p<0.05 vs CAST—NO FIBERS SAMPLES).

FIGS. 7A-7E are SEM images showing the quantification of the effects of linear grooves on anisotropy. The three factors that have been studied include: width, frequency and depth, as shown in FIG. 4C. The width resulted to be the most significant variable. FIG. 7A is a SEM image of an HMES groove with a width of 50 μm. FIG. 7B is a SEM image of an HMES groove with a width of 100 μm. FIG. 7C is a SEM image of an HMES groove with a width of 200 μm. A width of 200 μm induced mechanical and structural anisotropy. Experimental validation of the hypothesis included equi-stress biaxial testing (FIG. 7D) and digital image analysis of SEM images where fiber alignment was quantified with the orientation index (FIG. 7E).

FIGS. 8A-8C show examples of a potential application of a collagen fiber bundle orientation on heart valve. FIG. 8A is a numerical simulation of heart valve cusp deformation in systole. A 15° fiber orientation change affects radial deformation allowing for a better valve closure. ([*] Fan et al. “Optimal elastomeric scaffold leaflet shape for pulmonary heart valve leaflet replacement.” Journal of Biomechanics, 2013, 46(4):662-669) FIG. 8B depicts experimental evidence of collagen fiber bundle orientation in aortic valve ([**] Marom et al. “Fluid-structure interaction model of aortic valve with porcine-specific collagen fiber alignment in the cusps.” Journal of Biomechanical Engineering, 2013,135(10):101001-101006). FIG. 8C is a schematic for a numerical simulation of heart valve cusp deformation in systole. A 15° fiber orientation change affects fiber deformation at tissue level in terms of reducing strain concentration at the corners and allowing for higher deformation radially ([*] Fan et al. “Optimal elastomeric scaffold leaflet shape for pulmonary heart valve leaflet replacement.” Journal of Biomechanics, 2013, 46(4):662-669).

FIGS. 9A-9D show DCD and micro-grooves in controlling fiber orientation on engineered mitral valve processing. FIG. 9A shows the expected fiber orientation on a DCD mandrel with no pattern. FIG. 9B shows the expected fiber orientation on a grooved DCD mandrel. The base edge (the edge of the heart valve leaflet closest to the heart valve annulus) at the top of FIGS. 9A and 9B, and the apex (the edge of the heart valve leaflet most distal to the base or the heart valve annulus) at the bottom in FIGS. 9A and 9B. Fibers will follow the orientation of the pattern according to the “bridge effect” described in FIGS. 4A-4E. Orientation can be, therefore, controlled by micro-pattern regardless of the mandrel speed. As shown in FIG. 9B, the fibers are deposited in a convex pattern, curving outward, in relation to the base edge of the valve leaflet shape. FIGS. 9C and 9D show COMSOL numerical simulations showing the voltage distribution using a smooth (FIG. 9C) versus a grooved (FIG. 9D) mandrel (ΔV=24 kV).

FIGS. 10A-10E show quantitative fiber analysis on a patterned substrate. FIG. 7A show square hybrid electrospun micro pattern characterization via digital image analysis, where images show detected network and pore geometry. FIG. 10B is a graph depicting the normalized fiber diameter distribution (range 0.1-10 μm). FIG. 10C is a graph depicting the normalized orientation distribution (length weighted, range 0.5-1). FIG. 10D is a graph depicting the normalized pore size distribution (range 0-500 μm²). FIG. 10E is a graph depicting the normalized pore aspect ratio distribution (range 0-0.25).

FIGS. 11A-11D depict μ-DCD, HMES for cardiac tissue engineering. FIG. 11A is a computer aided designed (CAD) model of electrospinning mandrel with micro pattern for μ-DCD processing of TEHV. Grooves direction is based on the desired final fiber orientation. FIG. 11B is an image depicting that the electrodes in FIG. 11A can be combined with multi-degree of freedom robotic arms so that the collecting target is static and the injector is dynamic. This system allows for a more accurate control of the injector—target relative velocity and a more effective deposition on concave geometries. FIG. 11C is an example of HMES applied to TEHV, patterns can be prescribed for polymeric leaflets with different finalities including endothelization or enhanced tissue growth. FIG. 11D is an example of HMES applied to TEVG, patterns can be prescribed for graft tunica intima with different finalities including endothelialization and resistance to thrombosis.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values.

As used herein, the terms “comprising,” “comprise” or “comprised,” and variations thereof, are meant to be open ended. The terms “a” and “an” are intended to refer to one or more.

As used herein, the “treatment” or “treating” of a condition, wound, or defect means administration to a patient by any suitable dosage regimen, procedure, and/or administration route of a composition, device, or structure with the object of achieving a desirable clinical/medical end-point.

As used herein, the term “patient” or “subject” refers to members of the animal kingdom including but not limited to human beings and “mammal” refers to all mammals, including, but not limited to human beings.

A polymer composition is “biocompatible” in that the polymer and, where applicable, degradation products thereof, are substantially non-toxic to cells or organisms within acceptable tolerances, including substantially non-carcinogenic and substantially non-immunogenic, and are cleared or otherwise degraded in a biological system, such as an organism (patient) without substantial toxic effect. Non-limiting examples of degradation mechanisms within a biological system include chemical reactions, hydrolysis reactions, and enzymatic cleavage.

As used herein, the term “polymer composition” is a composition comprising one or more polymers. As a class, “polymers” includes, without limitation, homopolymers, heteropolymers, copolymers, and block copolymers, and can be both natural and/or synthetic. Homopolymers contain one type of building block, or monomer, whereas copolymers contain more than one type of monomer. The term “(co)polymer” and like terms refer to either homopolymers or copolymers.

A polymer “comprises” or is “derived from” a stated monomer if that monomer is incorporated into the polymer. Thus, the incorporated monomer that the polymer comprises is not the same as the monomer prior to incorporation into a polymer, in that at the very least, certain linking groups are incorporated into the polymer backbone or certain groups are removed in the polymerization process. A polymer is said to comprise a specific type of linkage if that linkage is present in the polymer. An incorporated monomer can be a “residue” of that monomer. A “macromer” or “macromonomer” refers to a monomeric subunit for incorporation into a (co)polymer, and can be a macromolecule that has at least one end-group which enables it to act as a monomer molecule. It may be a combination product of two or more smaller monomer residues.

As used herein, the “number average (Mn) molecular weight” is the total weight of a polymer divided by the total number of molecules of the polymer.

As used herein, a “moiety” is a part of a molecule, and can include as a class “residues”, which are the portion of a compound or monomer that remains in a larger molecule, such as a polymer chain, after incorporation of that compound or monomer into the larger molecule, or “functional groups”, which are specific substituents or moieties to which a characteristic chemical reactivity, non-covalent interactivity, physical characteristic, or other chemical or physical properties may be attributed.

As used herein, “alkyl” refers to straight, branched chain, or cyclic hydrocarbon groups including, for example, from 1 to about 100 carbon atoms, for example and without limitation C₁₋₃, C₁₋₆, C₁₋₁₀ groups, for example and without limitation, straight, branched chain alkyl groups such as methyl, ethyl, propyl, butyl, pentyl, hexyl, heptyl, octyl, nonyl, decyl, undecyl, dodecyl, and the like. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of straight alkyl groups include methyl, ethyl, propyl, butyl, pentyl, hexyl, heptyl, octyl, nonyl, and decyl. Branched alkyl groups comprise any straight alkyl group substituted with any number of alkyl groups. Non-limiting examples of branched alkyl groups include isopropyl, isobutyl, sec-butyl, and t-butyl. Non-limiting examples of cyclic alkyl groups include cyclopropyl, cyclobutyl, cyclopentyl, cyclohexyl, cycloheptyl, and cyclooctyl groups. Cyclic alkyl groups also comprise fused-, bridged-, and spiro-bicycles and higher fused-, bridged-, and spiro-systems. A cyclic alkyl group can be substituted with any number of straight, branched, or cyclic alkyl groups. “Substituted alkyl” can include alkyl substituted at 1 or more (e.g., 1, 2, 3, 4, 5, or even 6) positions, which substituents are attached at any available atom to produce a stable compound, with substitution as described herein. “Optionally substituted alkyl” refers to alkyl or substituted alkyl. “Alkylene” and “substituted alkylene” can include divalent alkyl and divalent substituted alkyl, respectively, including, without limitation, methylene, ethylene, trimethylene, tetramethylene, pentamethylene, hexamethylene, heptamethylene, octamethylene, nonamethylene, or decamethylene. “Optionally substituted alkylene” can include alkylene or substituted alkylene.

“Alkene or alkenyl” can include straight, branched chain, or cyclic hydrocarbyl groups including, e.g., from 2 to about 20 carbon atoms, such as, without limitation C₂₋₃, C₂₋₆, C₂₋₁₀ groups having one or more, e.g., 1, 2, 3, 4, or 5, carbon-to-carbon double bonds. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. A halo-alkenyl group can be any alkenyl group substituted with any number of halogen atoms. “Substituted alkene” can include alkene substituted at 1 or more, e.g., 1, 2, 3, 4, or 5 positions, which substituents are attached at any available atom to produce a stable compound, with substitution as described herein. “Optionally substituted alkene” can include alkene or substituted alkene. Likewise, “alkenylene” can refer to divalent alkene. Examples of alkenylene include without limitation, ethylene (—CH═CH—) and vinyl (H₂C═CH—) all stereoisomeric and conformational isomeric forms thereof. “Substituted alkenylene” can refer to divalent substituted alkene. “Optionally substituted alkenylene” can refer to alkenylene or substituted alkenylene. As used herein, “vinyl” is a H₂C═CH— group. “allyl” is a H₂C═CH—CH₂— group.

As used herein, “saturated” refers to a compound in which the atoms are linked by single bonds.

“Heteroatom” refers to any atom other than carbon or hydrogen, for example, N, O, P, and S. Compounds that contain N or S atoms can be optionally oxidized to the corresponding N-oxide, sulfoxide or sulfone compounds. “Heterocylic compound” refers to an organic compound in any embodiment described herein in which one or more carbon atoms in a cycloalkyl ring are substituted with any atom other than carbon or hydrogen, for example, N, O, P, or S. Where a cycloalkyl group is substituted with an O, forming one or more ether groups (—C—O—C) within the ring, the group can be referred to as “cycloether,” for example furanyl and tetrahydrofuranyl groups are C₄ cycloethers.

“Substituted” or “substitution” refer to replacement of a hydrogen atom of a molecule with one or more atoms or groups (substituents), such as, for example and without limitation: halogen, alkyl, alkoxy, alkylthio, trifluoromethyl, acyloxy, hydroxy, mercapto, carboxy, carbonyl, aryloxy, aryl, arylalkyl, heteroaryl, amino, alkylamino, dialkylamino, morpholino, piperidino, pyrrolidin-1-yl, piperazin-1-yl, thiol, nitro, nitrate, guanidinium, sulfo, sulfate, ═O (e.g., carbonyl), or other groups. “Halogen,” “halide,” and “halo” refers to —F, —Cl, —Br, and/or —I, and “halo-substituted”, refers to substitution of one or more atom or group, such as a hydrogen, with a halide. In aspects or embodiments, substituents may be, independently, and without limitation: ═O or a halogen.

As used herein, “siloxane” is a compound having one or more Si—O—Si linkages, e.g.,

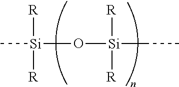

where each instance of R is, independently, an organic group or H, for example, straight or branched-chain C₁-C₄ alkyl, including methyl, ethyl, propyl, butyl, or phenyl C₁-C₄ alkyl, such as phenylmethyl or phenylethyl, optionally substituted with one or more halogen atoms. n typically varies from 1-2,000 with number average molecular weight (Mn) of, for example, about 1,000 to about 10,000, and increments therebetween. For polysiloxanes, n is greater than 1, e.g., from 10 to 200 or from 10 to 50, for example, for poly(dimethyl siloxane), n may range from 15 to 175 (approximating Mn=1,000 to 10,000), or from 15 to 35 (approximating Mn=1,000 to 10,000). Combination siloxanes include methylhydrogen, dimethylsiloxane, which includes a mixture of both methylhydrogensiloxyl and dimethylsiloxyl groups. In siloxanes, organic groups, such as without limitation, alkyl, haloalkyl, aryl, haloaryl, alkoxyl, aralkyl, and silacycloalkyl groups, and/or more reactive groups, such as alkenyl groups such as vinyl, allyl, propenyl, butenyl, pentenyl, hexenyl, heptenyl, octenyl, nonenyl, and/or decenyl groups may be attached to silicon atoms of the siloxane backbone in any combination. Polar groups, such as acrylate, methacrylate, amino, imino, hydroxy, epoxy, ester, alkyloxy, isocyanate, phenolic, polyurethane oligomeric, polyamide oligomeric, polyester oligomeric, polyether oligomeric, polyol, and carboxypropyl groups may be attached to silicon atoms of the siloxane backbone in any combination and in combination with any groups described herein. Siloxanes may be terminated with any useful group, for example and without limitation, alkenyl, and/or alkyl groups, such as a methyl, ethyl, isopropyl, n-propyl, allyl, or vinyl group, or any combination thereof. Other groups that may be used to terminate a siloxane include: acrylate, methacrylate, amino, imino, hydroxy, epoxy, ester, alkyloxy, isocyanate, phenolic, polyurethane oligomeric, polyamide oligomeric, polyester oligomeric, polyether oligomeric, polyol, carboxypropyl, and halo, e.g., fluoro groups.

Non-limiting examples of poly(dialkylsiloxanes) are polydimethylsiloxane (PDMS), e.g.,

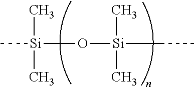

where n is a number of 1 or more, for example and without limitation, ranging from 1-10,000; diphenylsiloxane; diethylsiloxane; trifluoropropyl methyl siloxane; phenylmethylsiloxane; a copolymer of dimethylsiloxane with one or more of a diphenylsiloxane, a diethylsiloxane, a trifluoropropyl methyl siloxane, and/or a phenylmethylsiloxane, and an aminopropylmethylsiloxane-dimethylsiloxane. The poly(dialkylsiloxane)diol and the poly(dialkylsiloxane)diamine may comprise dimethyl siloxane.

As described herein, a “fiber” is an elongated, slender, thread-like, and/or filamentous structure. A “matrix” is any two- or three-dimensional arrangement of elements (e.g., fibers), either ordered (e.g., in a woven or non-woven mesh) or randomly-arranged. A matrix can be contiguous, as in the formation of a structure by molding, or can be an arrangement of particles and/or fibers. A matrix can be isotropic, that is, having one or more physical properties that has the same value when measured in different directions. A matrix can be anisotropic, that is, having one or more physical properties that have a different value when measured in a different direction. A matrix can be isotropic due to random orientation, alignment, or deposition of fibers, and can be anisotropic due to non-random orientation, alignment, or deposition of fibers. A matrix can have isotropic and anisotropic portions.

By “biodegradable” or “bioerodable,” it is meant that a polymer, once implanted and placed in contact with bodily fluids and tissues, will degrade either partially or completely through chemical reactions with the bodily fluids and/or tissues, typically and often preferably over a time period of hours, days, weeks, or months. Non-limiting examples of such chemical reactions include acid/base reactions, hydrolysis reactions, and enzymatic cleavage. The biodegradation rate of the polymer matrix may be manipulated, optimized, or otherwise adjusted, so that the matrix degrades over a useful time period. The polymer or polymers typically can be selected so that it degrades in situ over a time period to optimize development of and/or mechanical conditioning of the tissue. Generally, a bioerodable polymer degrades substantially completely within two years after implantation in a patient.

A number of biocompatible, biodegradable elastomeric polymers and (co)polymers are known and have been established as useful in preparing cell growth support matrices, for example are useful in preparation of a polymeric heart valve or blood vessel as described herein. Non-limiting examples of a bioerodible polymer useful in the devices described herein, include: a polyurethane, a polyester, a polyester-containing copolymer, a polyanhydride, a polyanhydride-containing copolymer, a polyorthoester, and a polyorthoester-containing copolymer. In one aspect, the polyester or polyester-containing copolymer is a poly(lactic-co-glycolic) acid (PLGA) copolymer. In other aspects, the bioerodible polymer is selected from the group consisting of poly(lactic acid) (PLA); poly(trimethylene carbonate) (PTMC); poly(caprolactone) (PCL); poly(glycolic acid) (PGA); poly(glycolide-co-trimethylenecarbonate) (PGTMC); poly(L-lactide-co-glycolide) (PLGA); polyethylene-glycol (PEG-) containing block copolymers; and polyphosphazenes. Additional bioerodible, biocompatible polymers include: a poly(ester urethane) urea (PEUU); poly(ether ester urethane)urea (PEEUU); poly(ester carbonate)urethane urea (PECUU); poly(carbonate)urethane urea (PCUU); a polyurethane; a polyester; a polymer comprising monomers derived from alpha-hydroxy acids such as: polylactide, poly(lactide-co-glycolide), poly(I-lactide-co-caprolactone), polyglycolic acid, poly(dl-lactide-co-glycolide), and/or poly(I-lactide-co-dl-lactide); a polymer comprising monomers derived from esters including polyhydroxybutyrate, polyhydroxyvalerate, polydioxanone, and/or polyglactin; a polymer comprising monomers derived from lactones including polycaprolactone; or a polymer comprising monomers derived from carbonates including polycarbonate, polyglyconate, poly(glycolide-co-trimethylene carbonate), or poly(glycolide-co-trimethylene carbonate-co-dioxanone). Those of skill will appreciate that biocompatible, biodegradable, elastomeric materials may be useful for heart valves as described herein.

In aspects, diamines and diols are useful building blocks for preparing the (co)polymer compositions described herein. Diamines as described above have the structure H₂N—R—NH₂ where “R” is an aliphatic or aromatic hydrocarbon, hetero-substituted hydrocarbon, or a hydrocarbon or hetero-substituted hydrocarbon, comprising aromatic and aliphatic regions. The hydrocarbon may be linear or branched. Examples of useful diamines are putrescine (R=butylene) and cadaverine (R=pentylene). Useful diols include polycaprolactone, multi-block copolymers, such as polycaprolactone-PEG copolymers, including polycaprolactone-b-polyethylene glycol-b-polycaprolactone triblock copolymers of varying sizes. Other building blocks for useful diols include, without limitation, glycolides (e.g., polyglycolic acid (PGA)), lactides, dioxanones, and trimethylene carbonates. Diisocyanates also may be used to prepare the polymer compositions described herein, having the general structure OCN—R—NCO, where “R” is an aliphatic or aromatic hydrocarbon or hetero-substituted hydrocarbon, a hydrocarbon or hetero-substituted hydrocarbon, comprising aromatic and aliphatic regions. The hydrocarbon may be linear or branched.

The polymer composition may comprise a biodegradable poly(ester urethane) urea (PEUU), poly(ether ester urethane)urea (PEEUU), poly(ester carbonate)urethane urea (PECUU), or poly(carbonate)urethane urea (PCUU). The composition may comprise a poly(ester-urethane)urea (PEUU). PEUU can be synthesized using putrescine as a chain extender and a two-step solvent synthesis method. For example, a poly(ester urethane) urea elastomer (PEUU) may be made from polycaprolactonediol and 1,4-diisocyanatobutane, with a diamine, such as putrescine as the chain extender. A suitable PEUU polymer may be made by a two-step polymerization process whereby polycaprolactone diol, 1,4-diisocyanatobutane, and putrescine are combined in a 1:2:1 molar ratio though virtually any molar feed ratio may suffice so long as the molar ratio of each monomer component is >0. In one aspect, the molar feed ratio of polycaprolactone diol plus putrescine is equal to that of diisocyanatobutane. A poly(ether ester urethane) urea elastomer (PEEUU) may be made by reacting polycaprolactone-b-polyethylene glycol-b-polycaprolactone triblock copolymers with 1,4-diisocyanatobutane and putrescine. In one aspect, PEEUU is obtained by a two-step reaction using a 2:1:1 reactant stoichiometry of 1,4-diisocyanatobutane:triblock copolymer:putrescine.

The composition may comprise a poly(ester carbonate urethane)urea (PECUU) or a poly(carbonate)urethane urea (PCUU) material. PECUU and PCUU are described, for example, in Hong et al., Tailoring the degradation kinetics of poly(ester carbonate urethane)urea thermoplastic elastomers for tissue engineering scaffolds, Biomaterials (2010) 31(15): 4249-58. PECUU is synthesized, for example, using a blended soft segment of polycaprolactone (PCL) and poly(1,6-hexamethylene carbonate) (PHC) and a hard segment of 1,4-diisocyanatobutane (BDI) with chain extension by putrescine. Different molar ratios of PCL and PHC can be used to achieve different physical characteristics. Putrescine is used as a chain extender by a two-step solvent synthesis method. In one example, the (PCL+PHC):BDI:putrescine molar ratio is defined as 1:2:1.

Implantable fiber matrix devices described herein can be formed by any useful electrodeposition method. The polymeric matrix may be electrodeposited, e.g., electrospun onto a target, such as a mandrel, and the resultant structure can be shaped post-deposition by cutting into shapes such as heart valve leaflet shapes or annular shaped structures (see, for example, U.S. Pat. Nos. 8,535,719 B2 and 9,237,945 B2, and United States Patent Application Publication No. 2014/0377213 A1, each of which is incorporated herein by reference for their disclosure of electrospinning methods, and variations on electrospun matrices, including synthetic and natural components). Heart valve leaflets and other anatomical structures often are, at least in part, anisotropic, and as such, the polymeric matrix that is used to prepare the fiber matrix may be deposited in an oriented manner, and is therefore anisotropic. Electrospinning and electrodeposition methods are broadly-known, and in electrodeposition, relative movement of the nozzles/spinnerets and target surface, e.g., by deposition onto a rotating mandrel, during electrodeposition can be used to produce an oriented pattern of fibers, though prior to the present disclosure, precise control of the electrodeposition anisotropy was difficult to achieve.

As is further broadly-known, more than one polymer composition can be electrodeposited concurrently, or in a desired order, to create a layered structure. Further, solutions comprising other polymers, ECM materials (e.g., ECM gel, or solubilized ECM), cell-culture medium, cells, such as stem cells including MSCs or ASCs, blood products, therapeutic agents, can be electrosprayed onto, or into the formed fiber structure, with variable deposition timing to create optimal layering or release of the soluble fraction. The properties of electrospun elastomeric matrices can be tailored by varying the electrospinning conditions. For example, when the biased target is relatively close to the orifice, the resulting electrospun mesh tends to contain unevenly thick fibers, such that some areas of the fiber have a “bead-like” appearance. However, as the biased target is moved further away from the orifice, the fibers of the non-woven mesh tend to be more uniform in thickness. Moreover, the biased target can be moved relative to the orifice. In certain embodiments, the biased target is moved back and forth in a regular, periodic fashion, such that fibers of the non-woven mesh are substantially parallel to each other. When this is the case, the resulting non-woven mesh may have a higher resistance to strain in the direction parallel to the fibers, compared to the direction perpendicular to the fibers. For portions of the described matrices, the biased target may be moved randomly relative to the orifice, so that the resistance to strain in the plane of the non-woven mesh is isotropic. The target can also be a rotating mandrel. In this case, the properties of the non-woven mesh may be changed by varying the speed of rotation. The properties of the electrospun elastomeric scaffold may also be varied by changing the magnitude of the voltages applied to the electrospinning system.

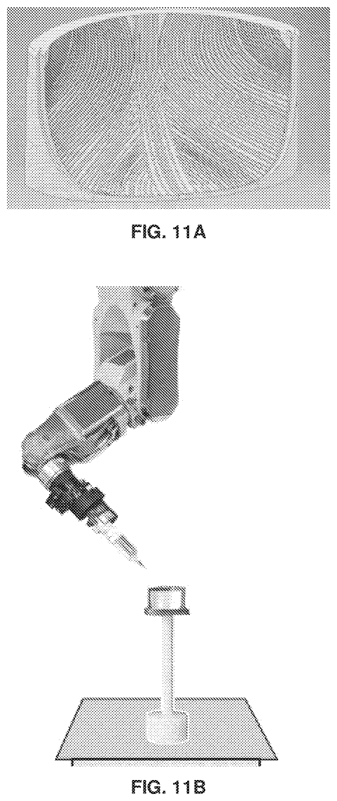

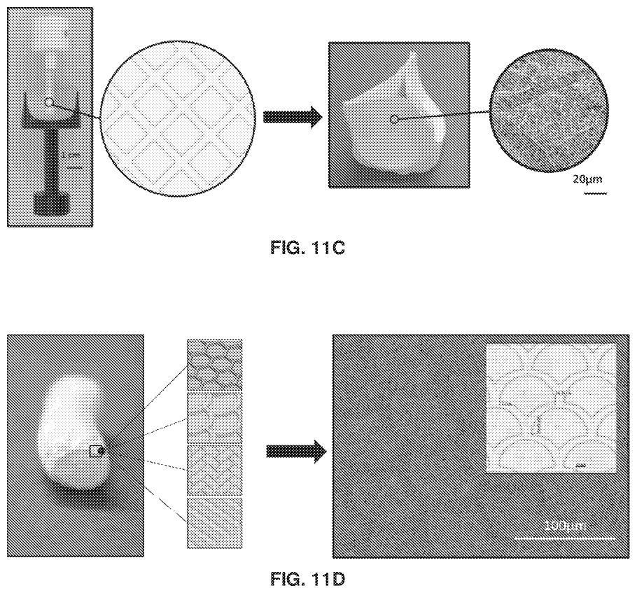

Although the above description describes movement of the target, the orifice/nozzle/spinnerette relative to the target (see, for example FIG. 11A-11D, described below). A CAD/CAM or similar computer-aided design file may be used or modified to guide the deposition of fibers onto a target using a controllable (e.g., robotic) arm or other controllable device under numerical control (computer numerical control or CNC), or any other suitable control process. Computer processes, instructions, applications, or software for use in CNC, such as for computer implemented printing are broadly-known and are adaptable to the processes described herein. Rates of deposition, distance from the target, and relative movement of the target and orifice, choice of polymer or other source material reservoir, among other parameters may be controlled.

Electrospinning may be performed using two or more nozzles, wherein each nozzle is a source of a different polymer solution. The nozzles may be biased with different biases or the same bias in order to tailor the physical and chemical properties of the resulting non-woven polymeric mesh. Additionally, many different targets may be used. In addition to a flat, plate-like target, a mandrel may be used as a target. When the electrospinning is to be performed using a polymer suspension, the concentration of the polymeric component in the suspension can also be varied to modify the physical properties of the elastomeric scaffold. For example, when the polymeric component is present at relatively low concentration, the resulting fibers of the electrospun non-woven mesh have a smaller diameter than when the polymeric component is present at relatively high concentration. One skilled in the art can adjust polymer concentrations to obtain fibers of desired characteristics. Useful ranges of concentrations for the polymer component include from about 1% wt. to about 15% wt., from about 4% wt. to about 10% wt., and from about 6% wt. to about 8% wt.

In electrospinning, polymer fibers may be deposited about the circumference of a mandrel and to generate a planar or substantially planar structure, the electrodeposited mat/matrix is cut substantially in the direction of the rotational axis of the mandrel, or in any manner to generate a useful topology, such as the shape of a heart valve, a heart valve leaflet, or a portion thereof. In use, more than one electrospun mats/matrices can be combined into a larger device by any useful means, such as by “sewing” using sutures, heat annealing, chemical annealing/cross-linking, etc., though it should be recognized that the method of attaching the two or more mats/matrices would have to be strong enough for the end use, e.g., to resist breakage, rupture, or herniation.

Although any form of spraying is expected to be effective, liquid, e.g., cell growth media, extracellular matrix (ECM) pre-gel, cells, a blood product, such as serum, plasma, or platelet-rich plasma, or a therapeutic composition, such as the soluble PCL ECM fraction described herein, may be electrosprayed. Electrospraying can be done before, after, or concurrently (intermittently or continuously) with the electrodeposition of polymer fibers, and can be conducted in an essentially identical manner.

The composition and structures according to any aspect described herein can also include additional components, such as an active agent, such as, without limitation, one or more of an antiseptic, an antibiotic, an analgesic, an anesthetic, a chemotherapeutic agent, an anti-inflammatory agent, a metabolite, a cytokine, a chemoattractant, a hormone, a steroid, a protein, or a nucleic acid, extracellular matrix (ECM) material(s), and/or cells. Such additions that may be incorporated, by themselves, or in combination with a suitable excipient, into the compositions described herein are described below.

The matrix described herein may comprise an extracellular matrix-derived (ECM) material, such as a gel (see, e.g., U.S. Pat. Nos. 8,361,503, and 8,691,276, the disclosures of which are incorporated herein by reference), as a base material in a polymeric composition/matrix (e.g., a composite), intermingled with the polymer matrix, and/or as a coating.

An “ECM material,” is a decellularized and/or devitalized material comprising or prepared from an extracellular matrix-containing tissue, and may not solely consist of a single, isolated and purified ECM component, such as a purified collagen preparation. A single, or more than one, component of ECM may be included in a polymeric matrix as described herein. Any type of tissue-derived material can be used to produce the ECM materials in the methods, compositions, and devices as described herein (see generally, U.S. Pat. Nos. 4,902,508; 4,956,178; 5,281,422; 5,352,463; 5,372,821; 5,554,389; 5,573,784; 5,645,860; 5,711,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666). The ECM material may be protease-solubilized, or otherwise-solubilized ECM material, such as ECM material that is acid-protease solubilized in acidic conditions—producing a reverse-gelling composition. The ECM material may be isolated from a vertebrate animal, for example and without limitation, from a mammal, including, but not limited to, human, monkey, pig, cow, and sheep. The ECM material can be prepared from any organ or tissue, including, without limitation, heart, urinary bladder, intestine, liver, esophagus, blood vessel, liver, nerve or brain, and/or dermis.

The polymer matrix or material may comprise one or more therapeutic agents. For example, at least one therapeutic agent is added to the polymer composition described herein before it is implanted in the patient or otherwise administered to the patient. Generally, the therapeutic agents include any substance that can be coated on, embedded into, absorbed into, adsorbed to, or otherwise attached to or incorporated onto or into a matrix as described herein. Non-limiting examples of such therapeutic agents include: anti-thrombogenic agents, growth factors, chemoattractants, cytokines, antimicrobial agents, emollients, retinoids, and steroids. Each therapeutic agent may be used alone or in combination with other therapeutic agents.

Active agents that may be incorporated into a matrix or material described herein include, without limitation, anti-inflammatories, such as, without limitation, nitro-fatty acids NSAIDs (non-steroidal anti-inflammatory drugs) such as salicylic acid, indomethacin, sodium indomethacin trihydrate, salicylamide, naproxen, colchicine, fenoprofen, sulindac, diflunisal, diclofenac, indoprofen sodium salicylamide, anti-inflammatory cytokines, and anti-inflammatory proteins or steroidal anti-inflammatory agents); antibiotics; anticlotting factors such as heparin, Pebac, enoxaparin, aspirin, hirudin, plavix, bivalirudin, prasugrel, idraparinux, warfarin, coumadin, clopidogrel, PPACK, GGACK, tissue plasminogen activator, urokinase, and streptokinase; growth factors. Other active agents include, without limitation: (1) immunosuppressants; glucocorticoids such as hydrocortisone, betamethasone, dexamethasone, flumethasone, isoflupredone, methylprednisolone, prednisone, prednisolone, and triamcinolone acetonide; (2) antibodies; (3) drugs acting on immunophilins, such as cyclosporine, zotarolimus, everolimus, tacrolimus, and sirolimus (rapamycin), interferons, TNF binding proteins; (4) taxanes, such as paclitaxel and docetaxel; statins, such as atorvastatin, lovastatin, simvastatin, pravastatin, fluvastatin, and rosuvastatin; (5) nitric oxide donors or precursors, such as, without limitation, Angeli's Salt, L-Arginine, Free Base, Diethylamine NONOate, Diethylamine NONOate/AM, Glyco-SNAP-1, Glyco-SNAP-2, S-Nitroso-N-acetylpenicillamine, S-Nitrosoglutathione, NOC-5, NOC-7, NOC-9, NOC-12, NOC-18, NOR-1, NOR-3, SIN-1, Hydrochloride, Sodium Nitroprusside, Dihydrate, Spermine NONOate, Streptozotocin; and (6) antibiotics, such as, without limitation: acyclovir, afloxacin, ampicillin, amphotericin B, atovaquone, azithromycin, ciprofloxacin, clarithromycin, clindamycin, clofazimine, dapsone, diclazuril, doxycycline, erythromycin, ethambutol, fluconazole, fluoroquinolones, foscarnet, ganciclovir, gentamicin, itraconazole, isoniazid, ketoconazole, levofloxacin, lincomycin, miconazole, neomycin, norfloxacin, ofloxacin, paromomycin, penicillin, pentamidine, polymixin B, pyrazinamide, pyrimethamine, rifabutin, rifampin, sparfloxacin, streptomycin, sulfadiazine, tetracycline, tobramycin, trifluorouridine, trimethoprim sulfate, Zn-pyrithione, ciprofloxacin, norfloxacin, afloxacin, levofloxacin, gentamicin, tobramycin, neomycin, erythromycin, trimethoprim sulfate, polymixin B, and silver salts such as chloride, bromide, iodide, and periodate.

Other drugs, active agents, or compositions that may promote wound healing and/or tissue regeneration may also be included in any material described herein.

Pharmaceutically acceptable salts, solvates, or prodrugs of any active agent (e.g., therapeutic agent or drug), bound to or otherwise combined with, or incorporated into a matrix or material described herein may also be employed. Pharmaceutically acceptable salts are, because their solubility in water is greater than that of the initial or basic compounds, particularly suitable for medical applications. These salts have a pharmaceutically acceptable anion or cation. Suitable pharmaceutically acceptable acid addition salts of the compounds of the invention include, without limitation, salts of inorganic acids such as hydrochloric acid, hydrobromic, phosphoric, metaphosphoric, nitric and sulfuric acid, and of organic acids such as, for example, acetic acid, benzenesulfonic, benzoic, citric, ethanesulfonic, fumaric, gluconic, glycolic, isethionic, lactic, lactobionic, maleic, malic, methanesulfonic, succinic, p-toluenesulfonic, and tartaric acid. Suitable pharmaceutically acceptable basic salts include without limitation, ammonium salts, alkali metal salts (such as sodium and potassium salts), alkaline earth metal salts (such as Mg and calcium salts), and salts of trometamol (2-amino-2-hydroxymethyl-1,3-propanediol), diethanolamine, lysine, or ethylenediamine. Pharmaceutically acceptable salts may be prepared from parent compounds by any useful method, as are well known in the chemistry and pharmaceutical arts.

Any useful cytokine or chemoattractant can be mixed into, mixed with, or otherwise combined with any composition as described herein. For example and without limitation, useful components include growth factors, interferons, interleukins, chemokines, monokines, hormones, and angiogenic factors. In certain non-limiting aspects, the therapeutic agent is a growth factor, such as a neurotrophic or angiogenic factor, which optionally may be prepared using recombinant techniques. Non-limiting examples of growth factors include basic fibroblast growth factor (bFGF), acidic fibroblast growth factor (aFGF), vascular endothelial growth factor (VEGF), hepatocyte growth factor (HGF), insulin-like growth factors 1 and 2 (IGF-1 and IGF-2), platelet derived growth factor (PDGF), stromal derived factor 1 alpha (SDF-1 alpha), nerve growth factor (NGF), ciliary neurotrophic factor (CNTF), neurotrophin-3, neurotrophin-4, neurotrophin-5, pleiotrophin protein (neurite growth-promoting factor 1), midkine protein (neurite growth-promoting factor 2), brain-derived neurotrophic factor (BDNF), tumor angiogenesis factor (TAF), corticotrophin releasing factor (CRF), transforming growth factors α and β (TGF-α and TGF-β), interleukin-8 (IL-8), granulocyte-macrophage colony stimulating factor (GM-CSF), interleukins, and interferons. Commercial preparations of various growth factors, including neurotrophic and angiogenic factors, are available from R & D Systems, Minneapolis, Minnesota; Biovision, Inc, Mountain View, California; ProSpec-Tany TechnoGene Ltd., Rehovot, Israel; and Cell Sciences®, Canton, Massachusetts.

Cells may be added to a material described herein, and/or are seeded thereon prior to introduction into a patient. Non-limiting examples of useful cells include: stem cells, progenitor cells, and differentiated cells; recombinant cells; cardiac valve cells and precursors thereof; mesenchymal progenitor or stem cells; endothelial cells; or valvular interstitial cells, including, without limitation, adipose-derived, placental-derived, umbilical cord derived, bone marrow derived, circulating (blood) derived, or skeletal muscle derived progenitor cells. Further examples of potentially useful cells include: venous and arterial (e.g. radial artery) endothelial cells, endothelial progenitor cells (EPC), mesenchymal stem cells derived EC isolated from a bone marrow biopsy or human umbilical cord-derived fibroblasts, and endothelial progenitor cells (See, e.g., Siepe et al., Stem Cells Used for Cardiovascular Tissue Engineering, European Journal of Cardio-thoracic Surgery, 2008, 34:242-247). In one aspect, the cells are autologous with respect to the patient to be treated. In another aspect, the cells are allogeneic with respect to the patient to be treated.

Cells, e.g., a patient's (autologous) cells or allogeneic cells, may be pre-deposited onto the matrix, and cultured ex vivo in a suitable bioreactor or culture vessel, as are known in the cell and tissue culture fields. A patient's blood or blood cells may be deposited onto the matrix, including suitable progenitor cells. Alternatively, or in conjunction with the ex vivo culturing, the device is implanted, and by virtue of contact with circulating blood cells and adjacent tissue, such as valve annulus tissue, the polymeric heart valve is infiltrated with and populated by the patient's cells. Heart valve leaflets have been generated in vivo in animals using single polymeric (PEUU, PCUU or PECUU) heart valve leaflet matrices sewn into heart valves.

Non-erodible polymers either do not erode substantially in vivo or erode over a time period of greater than two years. Compositions such as, for example and without limitation, PTFE, poly(ethylene-co-vinyl acetate), poly(n-butylmethacrylate), poly(styrene-b-isobutylene-b-styrene) and polyethylene terephthalate are considered to be non-erodible polymers. Other suitable non-erodible polymer compositions are broadly known in the art, for example, in stent coating and transdermal reservoir technologies. The matrices or materials described herein may comprise a non-erodible polymer composition.

Photolithography may be used to produce a template as described herein. Photolithography processes are broadly-known, such as soft photolithography using PDMS or similar materials, e.g., siloxanes (see, e.g., Chen et al. “Photolithographic surface micromachining of polydimethylsiloxane (PDMS).” Lab on a Chip, 2012, 12(2): 391-395). Soft photolithography methods are further described in the examples below.

### EXAMPLES

The most adopted technique to achieve control over a polymeric scaffold surface at the meso-scale is micro-molding. Micro-molding is utilized in tissue engineering to impose customized geometries on solid substrates and, in turn, to enhance a specific outcome such as cell proliferation or metabolism. Adequate substrate pattern and scale can considerably provide contact guidance and support cell growth. However, a solid geometry with a bulk porosity equal to zero is also characterized by a lower surface-to-volume ratio, lower permeability, and a slower degradation kinetics than an equivalent porous substrate with comparable surface pattern and identical chemistry. These factors all affect cellular proliferation and migration.

For example, a micro-fiber based substrate made of the same material and presenting the same surface topology at meso level would have higher surface-to-volume ratio, higher permeability and higher degradation kinetics. These features will provide more space for cell seeding, host cell recruitment, and proliferation. Moreover, the porous structure will offer a better exchange of nutrients and cell waste.

While effective, precise and reproducible micro-molding can only produce solid, non-porous geometry and can be hardly considered a processing technology scalable to the organ level. As such, surface topology modifications on centimeter (cm) size medical device surfaces such as a catheter, a stent or the rotor of a ventricular aided device cannot be obtained.

Several studies have proposed fibrous substrates with complex geometries Criscenti et al. (“3D screening device for the evaluation of cell response to different electrospun microtopographies”, Acta Biomaterialia, 2017, 55:310-322) and Vaquette et al. (“Increasing electrospun scaffold pore size with tailored collectors for improved cell penetration”, Acta Biomaterialia, 2011, 7:2544-57) proposed a squared fibrous surface, however in both cases the fabrication methods were not amenable to production of varied patterns, controlled and varied anisotropy, and complex shapes in the meso-scale, and especially under 50 μm. Rogers et al. (“A novel technique for the production of electrospun scaffolds with tailored three-dimensional micro-patterns employing additive manufacturing”, Biofabrication, 2014, 6:035003) proposed a deposition technology based on 3D-printed collector; however, the collector size was affected by the 3D printing spatial resolution that limited the application of this methodology at the meso-scale, further additive manufacturing is time-intensive as compared to soft lithography. Zhang et al. (“Fabrication of alignment polycaprolactone scaffolds by combining use of electrospinning and micromolding for regulating Schwann cells behavior”, Journal of Biomedical Materials Research Part A, 2018, 106:3123-3134) showed the ability to process fibrous patterned scaffold in a range of dimension close to the meso scale. Yet, that approach utilized only linear channels and ridges, with no indication of the ability to produce complex shapes. Dempsey et al. (“Micropatterning of electrospun polyurethane fibers through control of surface topography. Macromolecular Materials and Engineering”, 2010, 295:990-994) proposed a squared fibrous pattern processed on a PDMS mold with no indication that complex shapes (e.g., closed shapes other than regularly-arranged squares or rectangles, such as curved closed shapes or polygons having more than four vertices, such as pentagons or hexagons) could be effectively manufactured and serve as a substrate for production of a fibrous mat that recapitulates the patterns of the substrate. Finally, Cheng et al. (“Engineering the Microstructure of Electrospun Fibrous Scaffolds by Microtopography”, Biomacromolecules, 2013, 14:1349-1360) proposed a complex-shaped fibrous pattern, which however showed limited control in terms of the fiber density and orientation. In addition, the related in vitro data showed a limited cellular infiltration due to the fiber pore size.

### Example 1—Numerical Simulation

To better understand the mechanism of the principal driving force (electric force), the numerical simulation software COMSOL 5.0 was used to evaluate the behavior of the electro-static field of a typical electrospinning set-up and the behavior of the electro-static field of a patterned conductive collector.

### Materials and Methods

Starting from a simple set-up to calibrate the software, many simulations were successively run. Some configurations were investigated in a 2-dimensional domain while some others in a 3-D environment. All these analysis were conducted using electrostatic models in a stationary regimen.

After the definition of the geometry, the materials had to be defined. In each configuration, the conductive parts were described as aluminum or copper. For each component an electric potential was chosen. The external wooden box was always grounded. The mesh was always generated as “extremely dense” trying to use the smallest elements to reduce the error of the simulation. The needle potential was set at 12 kilovolts (kV) and the distance needle/collector was set at 8 cm.

### Results and Discussion

Different simulations indicated that the electrical field changes when different substrates are electro-spun. The substrate geometry modifies the equipotential lines close to the surface. As a consequence, the electric field adjusts its path to stay perpendicular to the equipotential line.

The electrical potential field close to the template surface was examined in the context of simulations (COSMOL, Palo Alto, CA) performed for grounded PDMS templates with different pyramidal geometries. The much higher field intensity over the posts compared to the bottom of the micro-wells indicates a higher electrostatic force at the top of the posts. This explains the fiber deposition on the posts and the fiber bridging over the micro-wells, and the almost conformal coverage of the posts by the fibers in the presence of a relatively more uniform field.

The first analysis was conducted to understand how the electric potential was distributed in the chamber. Focusing the attention on what happens on the collector, the PDMS mold was theorized to be nonconductive and nonconductive with an enhanced dielectric constant in two different simulations. The nonconductive PDMS mold εᵣ (relative dielectric constant) was 3.5 and the nonconductive PDMS mold with an enhanced dielectric constant εᵣ was 35.

The possibility to enhance the dielectric performance of a PDMS was investigated by Liu et al. using nanoparticles in a PDMS matrix (“Enhanced dielectric performance of PDMS-based three-phase percolative nanocomposite films incorporating a high dielectric constant ceramic and conductive multi-walled carbon nanotubes,” J. Mater. Chem. C, 2018, 6(40): 10829-10837). Liu et al. showed, as in previous works, that the dielectric constant was increased from εᵣ=3.5 to εᵣ=35, but the processability of the PDMS decreased due to the high fraction of fillers (Wang et al. “Significantly Enhanced Breakdown Strength and Energy Density in Sandwich-Structured Barium Titanate/Poly(vinylidene fluoride) Nanocomposites,” Adv. Mater., 2015, 27(42):6658-6663). In the work proposed by Liu et al., the dielectric constant was increased by a factor of 700, keeping good mechanical properties. Thus, increasing the dielectric constant of PDMS, the electrical potential can improve the transfer of the shape on a fibrous tissue.

These results indicate that the developed method can easily be used to fabricate fibrous structures with different characteristics, including fiber alignment, locally high/low porosity (density), and micro-wells of different dimensions for a variety of biological applications.

### Example 2—Evaluation of Electrospinning Parameters

The most influent electrodeposition parameters were evaluated: needle diameter, polymer concentration, electrical potential delta, needle to collector distance, and feed rate.

### Materials and Methods

Poly(ester urethane) urea (PEUU) Synthesis: In this process, 1,4-diisocyanatobutane (BDI), putrescine, and stannous octoate were used as received (Sigma-Aldrich). Poly(caprolactone) (PCL) (Molecular Weight (MW) 2000, Sigma-Aldrich) was dried under vacuum for 48 hours to remove the residual water. Dimethylsulfoxide (DMSO) was dried over 4 Angstrom (Å) molecular sieves. PEUU was synthesized using a two-step solution polymerization. The synthesis was carried out in a 250 milliliter (ml) three-necked round bottomed flask under a dry nitrogen atmosphere. The stoichiometry of the reaction was [2:1:1] of [BDI:PCL:Putrescine]. In the first polymerization step, a 15% (w/w) solution of BDI in DMSO was continuously stirred with a 25% (w/w) solution of PCL in DMSO, then stannous octoate was added. This mixture was allowed to react at 75 degrees Celsius (° C.) for 3 hours. The pre-polymer solution was cooled to room temperature before adding putrescine solution drop-wise under stirring. The reaction was continued at room temperature for 18 hours. The polymer solutions were precipitated in distilled water, and the wet polymer was immersed in isopropanol for 3 days to remove unreacted monomer. Finally, the polymer was dried under vacuum at 50° C. for 24 hours (Guan et al. “Synthesis, characterization, and cytocompatibility of elastomeric, biodegradable poly(ester-urethane)ureas based on poly(caprolactone) and putrescine,” J. Biomed. Mater. Res., 2002, 61(3):493-503; Jamadi, et al. “Synthesis of polyester urethane urea and fabrication of elastomeric nanofibrous scaffolds for myocardial regeneration,” Mater. Sci. Eng. C, 2016, 63:06-116).

Viscosity: The inherent viscosity of the polymer solution was evaluated using an Ubbelodhe viscometer. Over several months, the inherent viscosity was evaluated for 4 different batches of the same PEUU polymer over 15 days.

Photolithography: Photolithography is the process of transferring geometric shapes to the surface of a silicon wafer. The equipment used is the MLA100 Mask-less Aligner.

The steps involved in the photolithographic process were wafer cleaning, barrier layer formation, photoresist application, soft baking, mask alignment, exposure and development, and hard-baking. A circular 10-inch diameter silicon wafer was cleaned with combination of ethanol and isopropanol. After cleaning, a hexamethyldisilizane (HMDS) vapor prime was performed in order to create a barrier layer. Photoresist is usually function of the wanted depth since its sensitivity to UV allows dosing energy according to the preferred step depth.

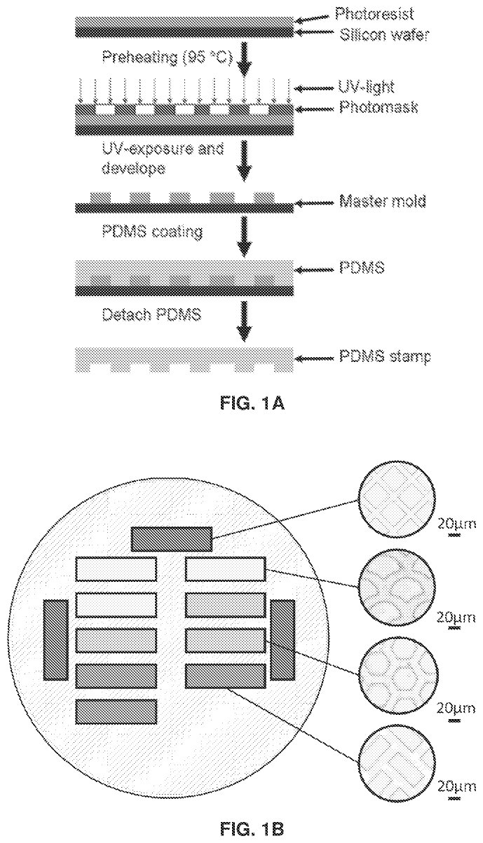

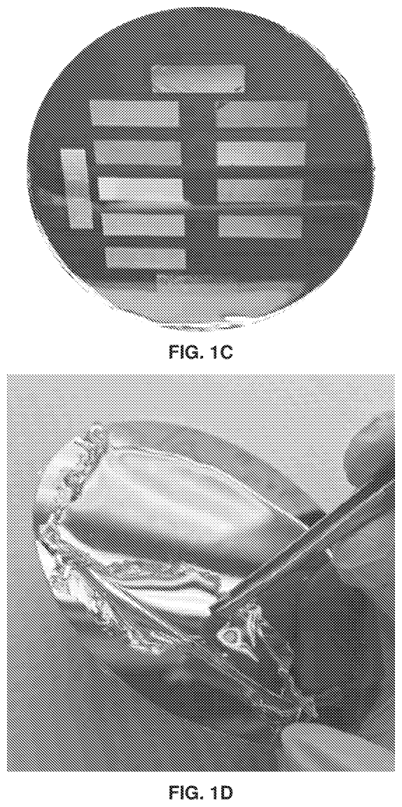

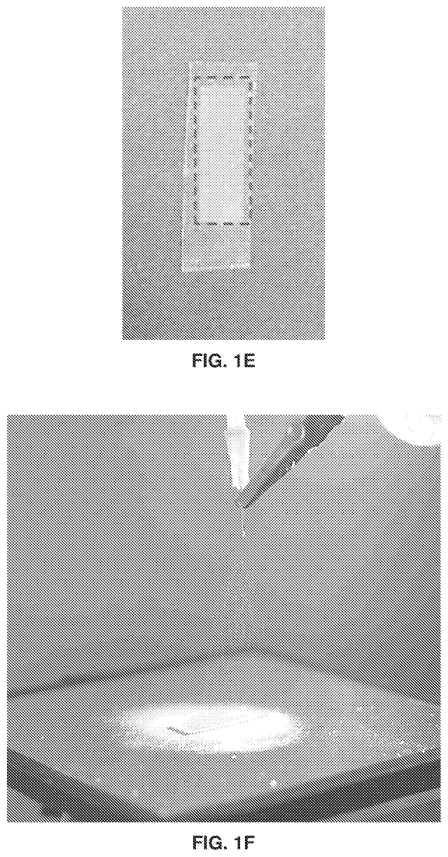

The photoresist used was SU8-2015. A first bake was done in order to enhance the adhesion of the photoresist to the wafer surface and ready the sample for the exposure. Exposition to an UV source was performed by a beam which can be either selective in the case of a mask-less aligner or a full beam in case of common mask photolithography process. After the exposure the development and the hard bake was done leading to the formation of the mold (FIG. 1A).

The HMDS vapor prime process cycle consisted of: vacuum/N₂ purge, HMDS process time, evacuation, and a final vacuum/N₂ purge. The total process cycle lasted 30 minutes and the pressure ranges from 0.1 to 760 Torr. After this step the photoresist coating was added.

Photoresist SU-2015 was spin-coated in 4 steps in order to obtain a gradual spreading:
1. 10 seconds of spinning at 600 rpm.
2. 60 seconds of spinning at 1200 rpm.
3. 60 seconds of spinning at 5000 rpm.
4. 10 seconds of spinning at 600 rpm.

The photoresist was selected considering the desired UV penetration depth. The wanted depth is 7 micrometers (μm) and for penetration less than 15 μm, SU8-2015 was selected. Once the wafer was coated, the next step was the positioning onto the mask-less aligner.

In order to communicate with the machine, a first design of the pattern had to be done in the format .GDS, precisely for that scope the patterns were drawn in KLayout.

KLayout is a freeware 2D layout viewer and editor that meets well the standards for the MLA. The program is defined by layers and cell: all same geometrical entities are defined under a defined layer while a cell is a hierarchy structured component. Hierarchy arises from the fact that KLayout offers two possible editing techniques. The first, direct editing is a simple user-friendly method by which editing of lines or simple polygons is performed. Secondly, a user-defined macro can be exploited for the automatization of sketches. Macro can be written either in Ruby or Python.

Since the final goal are engraved patterns on the electro-spun layer, the PDMS must have an embossed surface, which means that the original mold made by photolithography must be engraved (FIG. 1B). The length of the single patterned rectangles of FIG. 1B is 35 mm and their height is 7 mm. FIG. 1C shows the master mold obtained after the stereo-photolithography process.

Dosing Energy Test: Before submitting the whole pattern for the photolithography test, a dose test was made with a part of the pattern. Dosing energy was measured in Mega Joules per square centimeter (MJ/cm²) and it refers to the energy of the UV beam. In particular, a correct energy dose results in a clearer edge resolution and correct depth. For this study 200, 400 and 600 MJ/cm² were tested, and 600 MJ/cm² resulted in the best performance, which showed a depth of around 6-7 μm. After defining the drawing and the energy dose, the fabrications were submitted. After the MLA, the wafer mold was baked at 90° C. and developed with SU8 developer.

PDMS mold fabrication: PDMS was prepared by a combination of cross-linker and base at a ratio of 1:10 at room temperature. After the mix, the PDMS was poured onto the mold and successively exposed to vacuum at 20° C. in order to remove the bubbles, which influences the mechanical properties of the outcome (Kim et al. “Measurement of nonlinear mechanical properties of PDMS elastomer,” in Microelectronic Engineering, 2011, 88(8):1982-985). Finally, thermal curing was performed in an oven at 65° C. overnight.

The removal of the PDMS from the mold is a sensitive step of the process, especially in case of low thickness. This latter is influenced by the quantity of PDMS poured onto the mold and by the homogeneity of the deposition. Finally, the patterned PDMS was ready for further process steps or characterization (FIG. 1D). PDMS is then divided into several piece each containing a single pattern (FIG. 1E).

Photo sampling of electro-deposition: A region about 5 cm across near the vertex of the envelope cone was imaged with a lens that had a focal length of 86 mm and an f number of 1.0. The lens was placed about 50 cm from the jet to avoid disturbing the electrical field near the jet. The image produced by this lens was observed using a 12.5-75 mm, f 2.8 zoom lens on digital camera that shot pictures using the ISO parameter fixed at 100 and exposure time at 1/1000 seconds.

The light source was a 1500 Lumen LED lamp. An opaque plastic panel was used to project an image of the halogen lamp and its reflector onto the region occupied by the cone. The camera that was used to capture the pictures was a CANON D500. Image processing and analysis were done with Adobe Photoshop, Corell Photopaint and the software supplied with the electronic camera.

Controlled Parameters Chamber: A controlled parameter chamber was built to avoid that different experiments conducted in different days may be executed under different environmental parameters. The chamber was able to keep the temperature (T) and the Relative Humidity (RH %) constant.

Temperature control: The temperature control was implemented using a halogen lamp in the closed chamber. All the experiments were conducted at the temperature of 21° C. When the temperature was lower than 21° C., the lamp was switched on, waiting for the temperature to increase again. The temperatures were visualized instantly using the thermo-hygrometer Govee H5074. This device has a quick communication with a phone through the Govee app. Through this device, it is possible to know the temperature and the humidity in the chamber. All the recorded data was be sent by e-mail and analyzed with Excel or analogue software.

Relative humidity control: To increase the humidity rate in the chamber, a humidifier was used. Dry air was preventively mixed with moisturized air into a dedicated box and then was blown into the chamber with the right water rate. The addition of water makes the air colder, so it was necessary to control the parameters to reach the desired point. The relative humidity was maintained at 30%.

Process Parameters Choice: The electrospinning process is influenced by a large number of parameters. The following 5 parameters can be handled with rapid intervention: electric potential (kV); solution concentration (%); needle diameter (gauge, G); needle to collector distance (cm); and feed rate (milliliters per hour, ml/h). The following parameters in Table 1 were evaluated.

TABLE 1
Parameter choice for the electrospinning process optimization.

| Parameter | Value | |
|---|---|---|
| Polymer Concentration | 10% | |
| | 12% | |
| | 14% | |
| Needle Diameter (G) | 19 | G |
| | 21 | G |
| | 23 | G |
| Distance Needle to Collector | 6 | cm |
| | 9 | cm |
| | 12 | cm |
| Feed Rate | 0.4 | ml/h |
| | 0.8 | ml/h |
| Electrical Potential | 5 | kV |
| | 8 | kV |
| | 11 | kV |

The 5 parameters previously described were sorted by their difficulty to be changed. Since the change in the concentration of the polymer solution involves the replacement of the polymer solution itself and therefore a long downtime, this parameter was evaluated as the last to modify. Similarly, even the diameter of the needle is a parameter that implies the momentary stop of the apparatus. However, in this case the time necessary to modify the set-up is less than the change of polymer solution. For this reason, the change in the needle diameter was classified as the fourth parameter to be changed. Gradually decreasing the intervention difficulty, the last parameter that requires a physical change of the set-up, causing a further machine stop, is the distance of the collector from the needle. This parameter was therefore chosen as the third parameter to be modified. Considering that the change of the feed rate may be simply done by modifying the pump settings of the Harvard Apparatus, this parameter was evaluated as the second to be modified. Finally, for similar reasons, the modification of the electric potential was the first intervention parameter. In this case, in fact, it was sufficient to turn a knob on the voltage generator to obtain an applied potential different from the previous one.

Going further to the experimental planning, the 5 parameters previously sorted by their difficulty to be changed were divided in 3 principal tables organized by polymer concentration. The exemplary table for testing 10%, 12%, and 14% polymer solutions can found in Table 2. Each configuration was investigated three times to have a 3 element statistical sample. Therefore, 162 experiments per solution concentration were conducted for a total of 486 electro-depositions. Table 2. Experimental scheme for electrodepositing a specific % PEUU solution.

TABLE 2
Experimental scheme for electrodepositing
a specific % PEUU solution.

| Polymer | Needle | Needle- | Feed | Electrical |
|---|---|---|---|---|
| Concen- | Diameter | Collector | Rate | Potential |
| tration | (G) | Distance (cm) | (ml/h) | (kV) |
| % PEUU | 19 | 6 | 0.4 | 5, 8, and 11 |
| | | | 0.8 | 5, 8, and 11 |
| | | 9 | 0.4 | 5, 8, and 11 |
| | | | 0.8 | 5, 8, and 11 |
| | | 12 | 0.4 | 5, 8, and 11 |
| | | | 0.8 | 5, 8, and 11 |
| | 21 | 6 | 0.4 | 5, 8, and 11 |
| | | | 0.8 | 5, 8, and 11 |
| | | 9 | 0.4 | 5, 8, and 11 |
| | | | 0.8 | 5, 8, and 11 |
| | | 12 | 0.4 | 5, 8, and 11 |
| | | | 0.8 | 5, 8, and 11 |
| | 23 | 6 | 0.4 | 5, 8, and 11 |
| | | | 0.8 | 5, 8, and 11 |
| | | 9 | 0.4 | 5, 8, and 11 |
| | | | 0.8 | 5, 8, and 11 |
| | | 12 | 0.4 | 5, 8, and 11 |
| | | | 0.8 | 5, 8, and 11 |

Electrospinning: The patterned PDMS is positioned onto an aluminum plate, which is grounded. The syringe with a PEUU solution is positioned in a Harvard Apparatus to control the feed rate of the polymer solution. The needle is charged positively by means of a clamp and a voltage generator (FIG. 1G). The time of fabrication was 5 minutes.

PEUU solutions were also deposited onto a metallic plate having patterns with different geometries and surface roughness values.

Fluorescence Microscopy Analysis: After electrodeposition, the samples were observed using a normal fluorescence microscope. The sample was peeled-off and placed on a microscope slide.

Scanning Electron Microscopy Analysis: A JSM-6335F SEM (JEOL USA, Peabody, MA, USA) was used and main scoping parameters were: accelerating voltage 3 kV, emission current 12 microamps (μA), working distance 8 millimeters (mm). The samples having a pattern as confirmed by fluorescence microscopy were sputter coated in order to create a 4 nm layer of gold and palladium.

Image Analysis: The images collected for the envelope cone were analyzed by ImageJ.

### Results and Discussion

The measured inherent viscosity values were similar between the four PEUU solution batches. The inherent viscosity ranged between 1.24 and 1.33, and was found to not influence the electrodeposition process.

A solution concentration under 10% was determined to be too thin and generated an electrospray process instead of electrospinning. A solution concentration over 14% was too thick and lead to high fiber diameters and bead formation.

A needle diameter larger than 19 G lead to polymer drops during the spinning, while a diameter smaller than 23 G lead to clot formation and cyclic interruption of the process.

Needle to collector distances outside of the proposed ranges made the process unstable for different ranges of electric potential.

Feed rates of less than 0.4 ml/h lead clot formation and cyclic interruption of the process, while feed rates over 0.8 ml/h caused polymer drops on the collector during the process.

An electrical potential over 11 kV started to show separation of the envelope cone. When the electrical potential was under 5 kV, the Taylor cone was not observed and the spinning was not able to start.

During each fabrication, a picture of the envelope cone was taken as explained above. For each picture, the opening angle of the envelope cone (“conic angle”) was measured. This value was assumed to be a parameter to assess the rate of instability of a process. The first set of parameters were evaluated at a PEUU concentration of 12%.

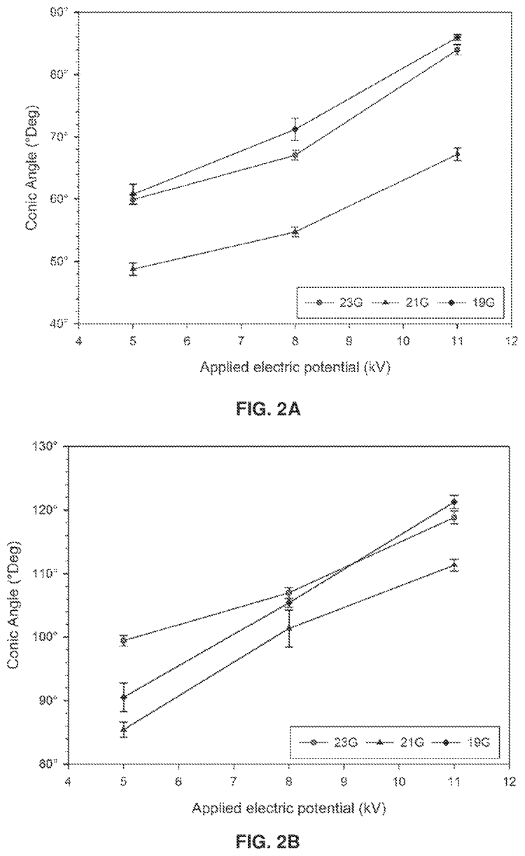

FIG. 2A shows the conic angle versus the applied voltage for a 12% PEEU solution at a feed rate of 0.4 ml/h through a 19 G, 21 G, and 23 G needle. FIG. 2B shows the conic angle versus the applied voltage for a 12% PEEU solution at a feed rate of 0.8 ml/h through a 19 G, 21 G, and 23 G needle. The conic angle tends to increase with the voltage. Moreover, an interesting result is related to the feed rate. In fact, as FIGS. 2A and 2B show, the larger the feed rate, the larger the conic angle. The size of the needle did not show a particular trend. Therefore, it was determined that the needle diameter has no influence on the conic angle.

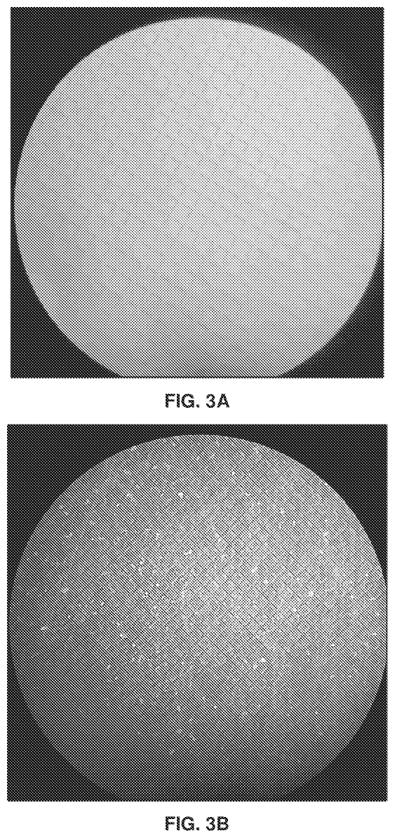

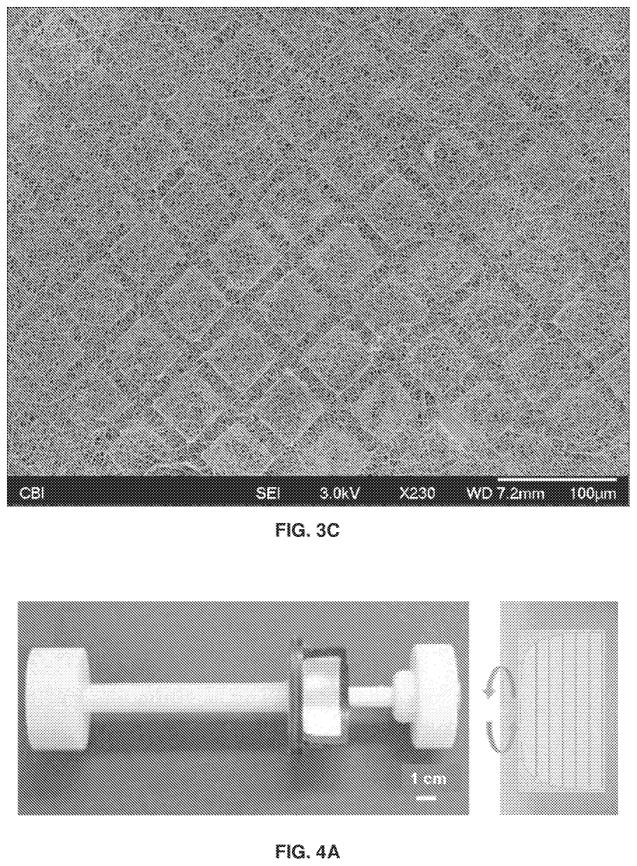

The electrodeposition process was able to replicate the shape of the PDMS mold (FIG. 3A). The parameters used to deposit a sample that replicated the shape of the mold were: a 12% PEUU solution; a 21 G needle; a 0.4 ml/h feed rate; a 9 cm needle to collector distance; a 11 kV electrical potential; a temperature of 21° C.; a relative humidity of 30%; and a 5 minute fabrication time. The fibrous patterned tissue was observed by fluorescence microscopy (FIG. 3B) and then confirmed by SEM (FIG. 3C).

The electrodeposition process was able to replicate the shape of the metallic plate having patterns with different geometries and surface roughness values. The fibrous patterned tissue was observed by fluorescence microscopy at a wave distance of 500 μm.

### Example 3

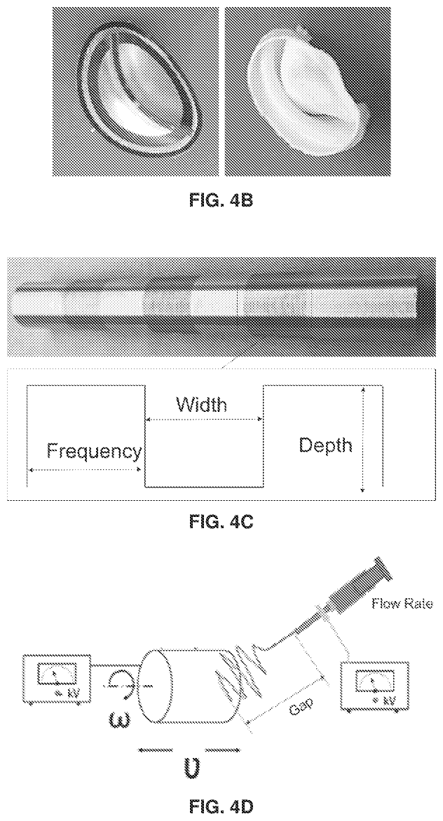

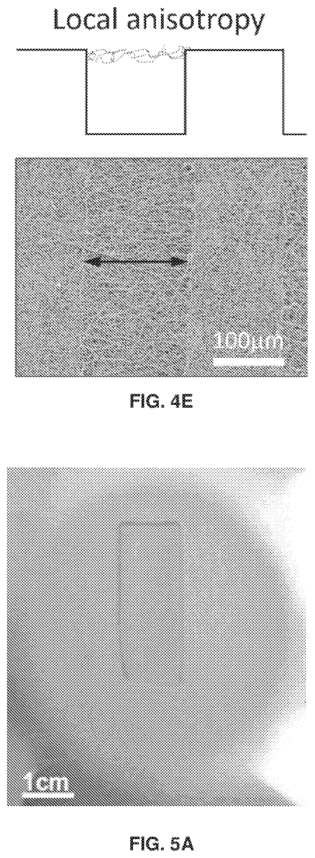

The invention combines the benefits of micro-fiber deposition with micro-molding for biomaterials surface processing. The technique is referred to herein as Hybrid Micro-molding Electrodeposition Surface (HMES) processing as it is composed of two main elements: a polydimethylsiloxane (PDMS) mold obtained by stereolithography or a micro-machined conductive material mold and a micro-fiber deposition method such as melt electrowriting, electrospinning, or jet spinning. Exemplary processing steps are summarized in FIGS. 1A-1F and 4A-4E.

Both methods (micro-molding and micro machining) can be used to customize collectors and/or electrodes, normally used in every polymeric fiber deposition technique regardless of the specific physical principle driving the filament creation (e.g., extrusion, voltage gap, mass acceleration). The overall concept is depositing a fiber network on top of a prescribed mesoscopic pattern so that cells will interact with such a pattern at the meso scale but will interact with a fibrillar substrate that allows for proper nutrient and waste exchange.

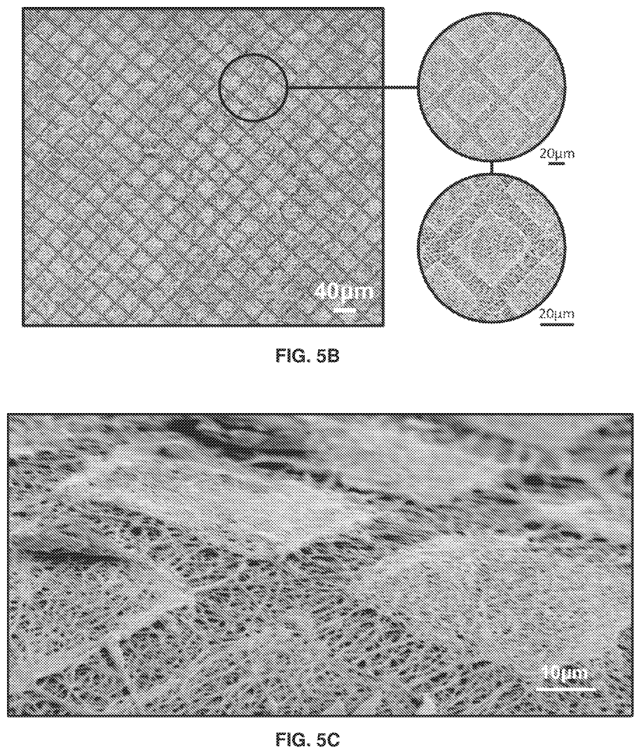

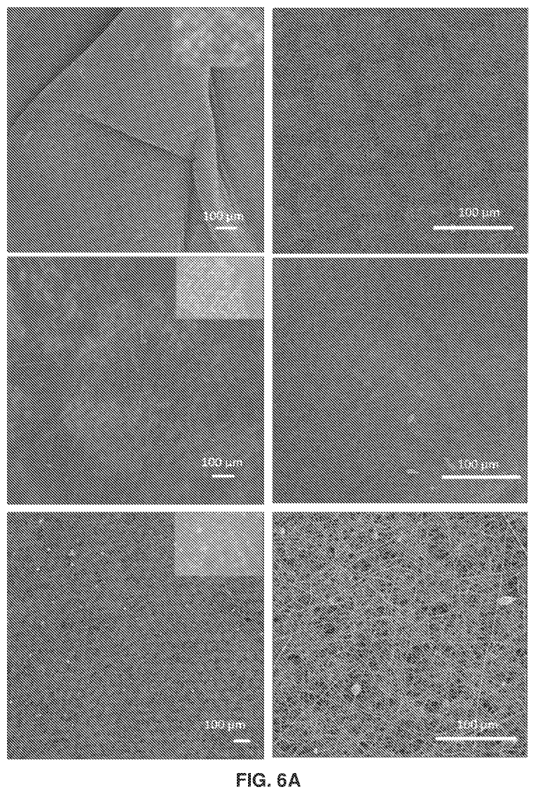

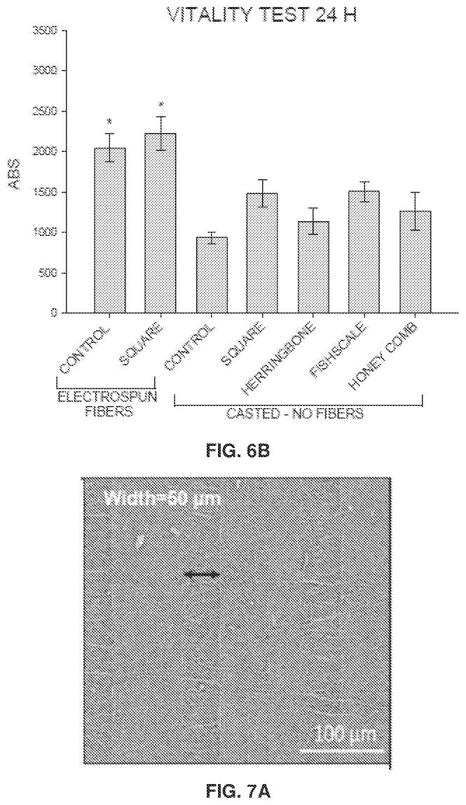

The substrate interfaces processed by HMES can replicate mechanics and topological features of native tissue. The full process is illustrated in FIGS. 1A-1F and 5A-5C. An example of how this process is applicable to cell manipulation over large surfaces (cm) is provided in FIG. 6A. Vascular Smooth Muscle Cells (VSMCs) proliferation was significantly higher in fibrous substrates is and it was the highest in HMES processed samples (FIG. 6B).

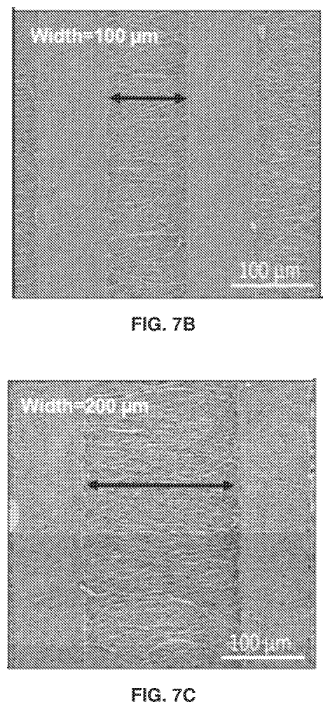

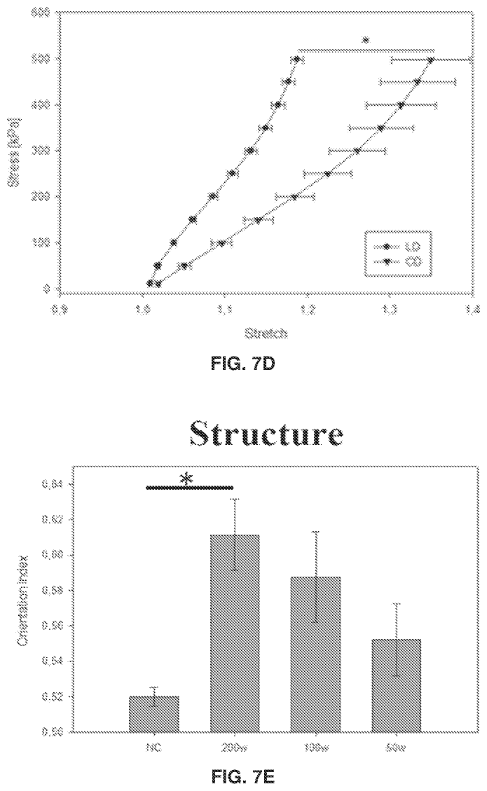

A second aspect of the invention is the induction local structural anisotropy on a polymeric substrate or scaffold. Dynamic collectors are used in melt electrowriting, electrospinning, jet spinning, and double component deposition (DCD). As an example, in the specific case of DCD for heart valve prosthesis processing (FIGS. 4A-4E), the high rotational speed of the collector produces a preferential direction of the fiber flocculation. Higher rotational speeds produce a higher tangential velocity and a higher number of fibers aligned with the circumferential direction of the collector/mandrel. In brief, higher alignment, and therefore structural and mechanical anisotropy, is a linear function of the collector radius and the angular speed. This element becomes a significant limiting factor when depositing on collectors with small radii or with concavity. In addition, air movement generated by the rotation of the collector disturbs the fiber deposition and negatively affects control of anisotropy. As shown herein, these issues may be circumvented by combining micro-grooves and static electro-deposition. Micro-grooves on a conductive material can induce local anisotropy by localizing maximum voltage distribution. Each edge of the groove machined in the collector will function as a preferential line for the fiber flocculation. As shown in FIGS. 4A-4E, this will generate local alignment over a direction perpendicular to the micro-groove and as a consequence local anisotropy at structural and mechanical level (FIGS. 7A-7C). FIG. 4A, depicts a polygonal cross-sections, such as where the ridges comprise a longitudinally-extending peak and a plurality of longitudinally-extending edges on each side of the peak and meeting the peak at edges forming an angle between the peak and walls. The angle may range from 45° to 135°, e.g., from 85° to 95°, e.g., approximately 90° (squared).

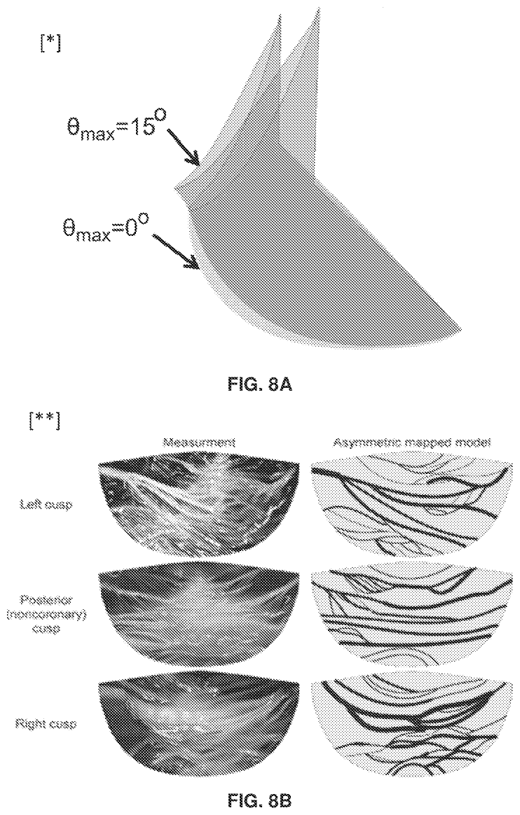

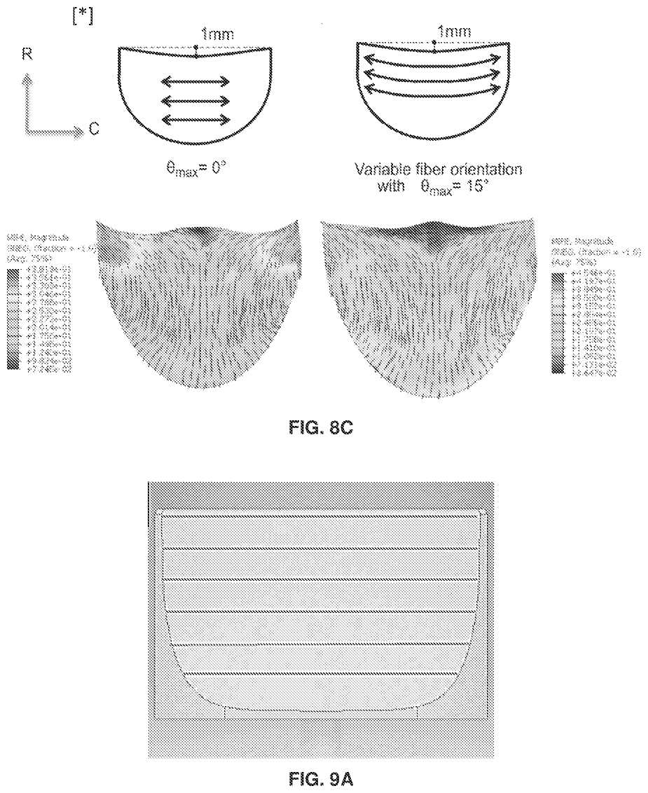

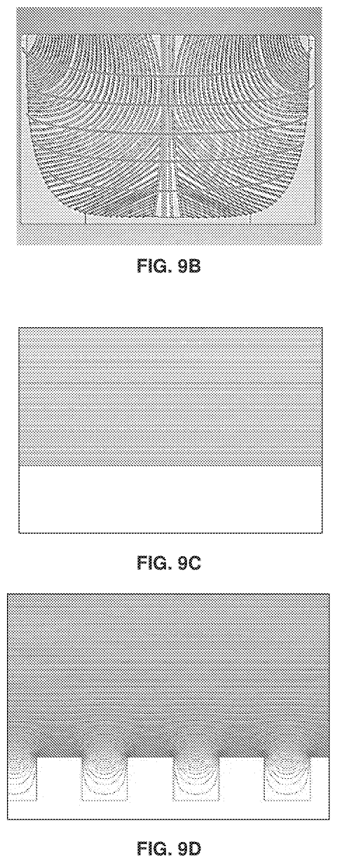

Mandrel or complex collector surfaces for electrodeposition can be modified by machining, stamping or micro-molding. An example of how this can be applied to tissue engineered heart valve is provided in FIGS. 4A-4E, 6A-6B, 7A-7E, 8A-8C, and 9A-9B. More specifically, this notion can be utilized to induce alignment but also to control fiber orientation to reproduce, for instance, collagen fiber architecture in native valves (FIGS. 8A-8C).

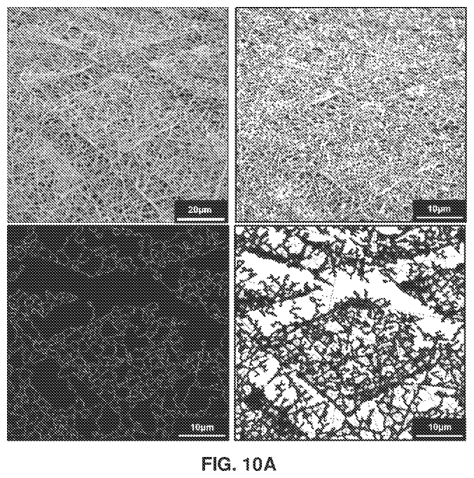

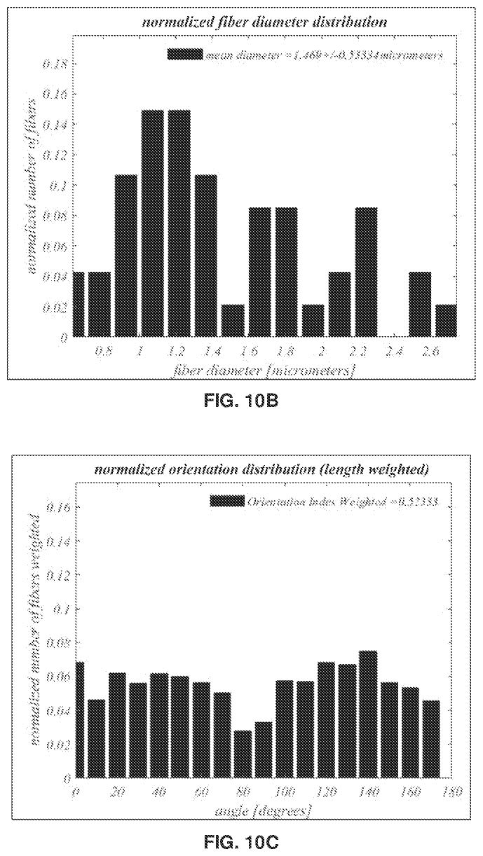

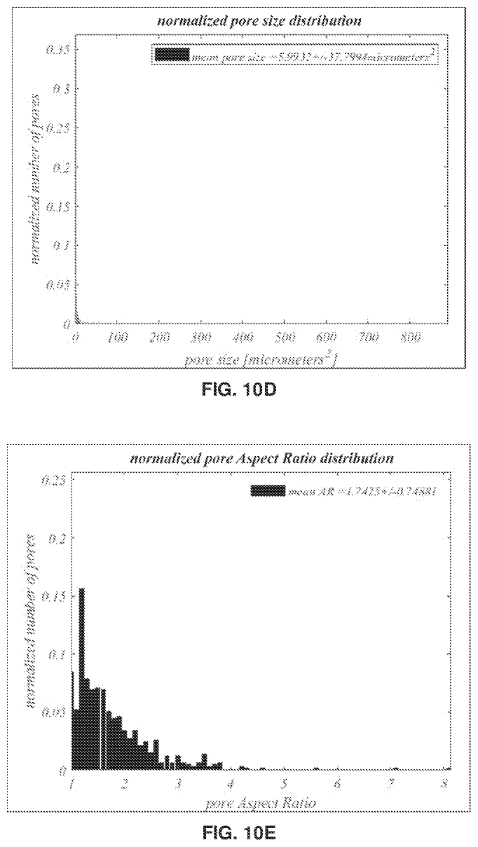

Ranges for fiber diameter, pore size, fiber alignment and pore shape are provided in FIGS. 10B-10E. While the meso-scale pattern is dictated by the groove geometry, conventional parameters in electrodeposition (e.g., voltage gap, polymer viscosity, gap distance) can control fiber and pore microscopic geometry. In essence, DCD is extended to the micro-scale by utilizing local voltage manipulation to control fiber deposition. In light of this principle we named this technique μ-DCD. Last, in order to enhance the control of fiber deposition and adapt the nozzle kinematics to any geometry of the collector, both HMES and μ-DCD can be combined with a robotic CAD-CAM system able to fine tune motion, speed and acceleration of the polymer source.

The elements of innovation introduced by HMES and μDCD (FIGS. 1A-1F, 4A-4E, 5A-5C, 6A-6B, 7A-7E, 8A-8C, 9A-9D, 10A-10E, and 11A-11D) can be summarized as follows:
A. Capacity to process polymeric scaffolds or substrate with complex patterns at the meso scale and micro fiber by combining micro-molding and micro-fiber deposition (FIGS. 1A-1F and 5A-5C).
B. Capacity to manipulate cell biology with HMES processed substrate (FIGS. 6A and 6B).
C. Capacity to tune pore size (pore size range 5 to 500 μm), fiber diameter (diameter range 0.1-10 μm), fiber orientation/alignment (orientation index range 0.5-1), pore aspect ratio (aspect ratio 0-0.25)(FIGS. 10B-10E).
D. Capacity to induce and modulate local anisotropy on 2D or 3D collector (FIGS. 4A-4E, 7A-7E, 8A-8C, 9A-9D).
E. Capacity to combine robotic CAD CAM system with both HMES and μDCD static collectors for enhanced control on fiber deposition (FIGS. 11A-11D).
F. Capacity to control fiber deposition at the meso scale with printed grooves of materials with higher or lower electrical conductivity of the collector material case.

### Materials and Methods

Design of the patterns: The design of the patterns has been done considering the interest in the investigation of different shape in the circularity-elongation matrix as well as sizes in term of area. Additionally the pattern must be biologically efficient given cells adaptation constrains and limitations. In order to meet these last requirements, cues have been taken from nature and its inspired geometries.

The selected patterns, which are the finished products of the whole fabrication chain, were engraved squares, honeycombs, herringbones and fish scales.

These patterns were designed using two major parameters: circularity and elongation.

Circularity is defined as a parameter that indicates the resemblance of a geometry to a circle. Quantifying circularity of a geometrical entity has been the goal of different studies (Olson “Particle Shape Factors and Their Use in Image Analysis-Part 1: Theory,” J. GXP Compliance, 2011, 15(3):85; Li et al. “An efficient measure of compactness for two-dimensional shapes and its application in regionalization problems,” Int. J. Geogr. Inf. Sci., 2013, 27(6):1227-1250) where the definition used is the below:

$$C = 4 π \frac{A}{{( {P}_{c o n v e x} )}^{2}}$$

where C is the circularity, A is the area of the shape, Pconvex is the perimeter of the convex shape that include the whole single pattern. By definition circularity is equal to 1 for the circle and less than 1 for the other entities. Elongation is the ratio between the length and width of the object bounding box (Stojmenović et al. “New measure for shape elongation,” in Lecture Notes in Computer Science (including subseries Lecture Notes in Artificial Intelligence and Lecture Notes in Bioinformatics), 2007):

$$E = \frac{{Length}_{box}}{{width}_{box}}$$

as circularity, elongation is a parameter less or equal to 1.

Square patterns are isotropic, space-optimized and less circular when compared to honeycombs. Herringbones are space-optimized, less circular and oriented along the flow direction. Similarly, fish scales are flow-oriented but are more circular and non-space optimized, which means that the distance of the wall is not the same along the pattern. The choice of this distance and depth has been taken considering the goal of a confluent cell layer. Because increases in depth with the same width alters the cell layer integrity (see, Uttayarat et al. “Topographic guidance of endothelial cells on silicone surfaces with micro- to nanogrooves: Orientation of actin filaments and focal adhesions,” J. Biomed. Mater. Res. —Part A, 2005, 75(3): 668-680; Uttayarat et al. “Micropatterning of three-dimensional electrospun polyurethane vascular grafts,” Acta Biomater., 2010 6(11):4229-4237; Bachmann et al. “Honeycomb-structured metasurfaces for the adaptive nesting of endothelial cells under hemodynamic loads,” Biomater. Sci., 2018, 6(10): 2726-2737)), an exemplary average distance between walls may be about 10 μm, and the depth about 7 μm.

Photolithography: The photolithography process was followed as described in Example 2.

PDMS mold fabrication: The method for PDMS mold fabrication was followed as described in Example 2.

Poly(ester urethane) urea (PEUU) Synthesis: PEUU was synthesized according to the method described in Example 2.

Electrospinning: The PDMS pattern was positioned onto an isolated aluminum plate. A 20 ml polypropylene syringe was fed by a 12% (w/w) PEUU in hexafluoroisopropanol (HFIP) was positioned in a Harvard apparatus in order to control the feed rate of the polymer which in this study was kept constant at 0.8 ml/h. The 23 G needle was charged positively by means of a clamp and a voltage generator. The voltage applied to the needle was 22 kV, while the aluminum plate was grounded.

The PDMS patterned mold was positioned on the top of the aluminum plate and then the electrodeposition was started. Every deposition was conducted for a time equal to 10 minutes per sample. The sample was dried for another 10 minutes in a vacuum oven at 20° C., and then it was extracted. (FIG. 5C).

Cell proliferation on micro-patterned films and scaffolds: Cell proliferation was evaluated on electrospun control and square samples and on square-, herringbone-, fish-scale-, and honeycomb-casted samples (FIGS. 6A-6B). Rat vascular smooth muscle cells (VSMC) were cultured in Dulbecco's modified eagle's medium (DMEM) with 10% fetal bovine serum (FBS) and 1% penicillin/streptomycin at 37° C. and 5% carbon dioxide (CO₂). Before cell seeding, scaffolds were sterilized in 70% of ethanol 3 times for 15 minutes each, 3 times in phosphate saline buffer (PBS) for 15 minutes each and finally 20 minutes under the effect of ultraviolet light. For the test, VSMC, with a number of density 12×10⁴/50 μl in DMEM, were seeded on top of each scaffold for 4 hours at 37° C. After unattached cells were washed out with PBS and finally cultured. Then, 24 hours later, the samples were washed with PBS and the vitality test was performed. The Alamar Blue assay was carried out according to manufacturer's instructions: 150 μl of Alamar Blue solution 1:10 in fresh DMEM were added to each scaffold. Following 1-hour incubation. Alamar Blue fluorescence was quantified using a fluorescence excitation wavelength of 560 nm and an emission of 590 nm.

Mandrel design for double component fiber deposition (DCD): The DCD mandrel model was developed in Solidworks (Waltham, MA, USA) and consisted of two components. The mitral heart valve-shaped conductive component was obtained by machining of aluminum alloy, and the non-conductive component was made by injection molding of acrylonitrile butadiene styrene (ABS) (FIGS. 4A and 4B). The heart valve shaped collecting surface was connected with a high voltage generator, and the “shield” component was mechanically connected to the motor providing the rotation. A second motor controlled the translational rastering speed (Amoroso et al. “Elastomeric electrospun polyurethane scaffolds: The interrelationship between fabrication conditions, fiber topology, and mechanical properties,” Adv. Mater., 2011, 23(1):106-111; Amoroso et al. “Microstructural manipulation of electrospun scaffolds for specific bending stiffness for heart valve tissue engineering,” Acta Biomater., 2012, 8(12):4268-4277). The conductive component diameter was selected to be 40 mm based on echocardiography data by Ring et al. (“Dynamics of the tricuspid valve annulus in normal and dilated right hearts: A three-dimensional transesophageal echocardiography study,” Eur. Heart J. Cardiovasc. Imaging, 2012, 13(9):756-762) of the anterior-posterior diameter D and septal-lateral diameter of human tricuspid valves throughout the cardiac cycle. The height (h) was calculated based on the diameter utilizing a h/D ratio of 1.6 utilized in the Sapien 3 prosthesis (Carpentier-Edwards, Irvine, CA).

Double component fiber deposition (DCD) processing conditions: PEUU was synthesized as described above and biodegradable valves were fabricated with electrospinning using the developed mandrel for DCD and the following process variables: polymer voltage 11 kV, mandrel voltage −5 kV, polymer gap 15.5 cm, polymer flow rate 1.5 mL/hr, polymer 12% w/v in HFIP, mandrel tangential velocities of 0.3-3 meters per second (m/s), mandrel rastering velocities 0-2.5 centimeters per second (cm/s).

Cylindrical electrode: The four cylindrical electrodes were machined in order to create the wanted grooves geometry. Four different CAD models were created for each rod in order to specify the dimension of each groove section. Each rod was composed by three different sections in which just one parameter among depth, width and frequency was changed, the other two were kept constant. (FIG. 4C).

Each groove section was spaced out by a smooth zone. In both the extremity of the rod, rubber rings were attached in order to clamp the electrode to the electrospinning device.

A table with all the different microgrooves dimension expressed in μm is reported for each electrode:

TABLE 3
The different microgrooves dimension expressed in um for each electrode.

| | Sections | | |
|---|---|---|---|
| | A | B | C |
| | Depth-width-frequency | Depth-width-frequency | Depth-width-frequency |
| Square | 50-50-50 | 100-100-100 | 150-150-150 |
| Depth | 50-100-100 (50d) | 100-100-100 (100d) | 200-100-100 (200d) |
| Width | 100-50-100 (50w) | 100-100-100 (100w) | 100-200-100 (200w) |
| Frequency | 100-100-50 (50f) | 100-100-100 (100f) | 100-100-200 (200 f) |

Numerical modeling: The numerical simulation software COMSOL 5.0 was used to analyze different configurations of the electrostatic field around the needle and the patterned collector. Imposing the boundary condition of a −6 kV negative potential on the collector and a 18 kV positive potential on the needle. A conductive aluminum plate was used during the analysis while the empty spaced was modeled using the integrated material AIR. (FIG. 8C).

Quantitative fiber analysis on a patterned substrate: Scattering electron microscopy (SEM) was used to study the micropattern architecture as described above in Example 2. An image analysis algorithm previously developed (Olson) was applied to the micropattern samples and fiber diameter distribution, orientation distribution, pore size distribution, and aspect ratio was quantified.

While several examples and embodiments of the template, valve construct, and treatment methods are shown in the accompanying figures and described hereinabove in detail, other examples and embodiments will be apparent to, and readily made by, those skilled in the art without departing from the scope and spirit of the invention. For example, it is to be understood that this disclosure contemplates that, to the extent possible, one or more features of any embodiment can be combined with one or more features of any other embodiment. Accordingly, the foregoing description is intended to be illustrative rather than restrictive.

1. A method of making a polymer matrix structure with controlled fiber alignment, comprising, electrodepositing polymer fibers from an electrodeposition nozzle either onto a surface of a target comprising a non-homogenous pattern of alternating ridges and valleys extending generally perpendicularly to a fiber alignment direction to produce an aligned electrical field for depositing the polymer fibers in the fiber alignment direction, wherein a periodicity of the alternating ridges and valleys is optionally 500 μm or less and/or the valleys have a depth of 15 μm or less, or onto a surface of a siloxane target comprising a siloxane composition, the target comprising a repeated three-dimensional pattern or tessellation of raised shape with a periodicity of less than 500 μm.

2. The method of claim 1, wherein the target and/or surface of the target is conductive.

3. The method of any one of claim 1, wherein the target comprises a siloxane composition comprising the plurality of alternating ridges and valleys.

4. (canceled)

5. The method of claim 1, wherein the target comprises a siloxane composition comprising a conductor.

6. The method of claim 5, wherein the conductor comprises conductive particles, or the siloxane is doped with a conductive material.

7. (canceled)

8. The method of claim 5, wherein the conductor comprises polyaniline or a conductive carbon allotrope.

9. (canceled)

10. The method of claim 1, wherein the target further comprises a conductive layer deposited over at least a portion of the surface of the target.

11. The method of claim 1, wherein the surface of the target comprising the pattern of alternating ridges and valleys is metallic.

12. The method of claim 1, wherein at least a pair of adjacent ridges have polygonal cross-sections.

13. The method of claim 1, wherein one or both of the target and the electrodeposition nozzle move in a direction relative to each other during the electrodeposition, and at least a portion of the ridges and valleys of the target are not longitudinally extended perpendicular to a direction of relative movement of the target and the electrodeposition nozzle, thereby depositing a fiber in a direction not parallel to the direction of relative movement of the target and the electrodeposition nozzle.

14. The method of claim 1, wherein the target is a rotating mandrel or the target is static, and the electrodeposition nozzle is moved relative to the target.

15. (canceled)

16. The method of claim 1, wherein the target comprises a pattern of ridges and valleys configured to produce a fiber matrix in the size and shape of a heart valve leaflet having a base edge and an apex, and wherein the ridges and valleys are arranged to produce a convex arrangement of fibers with respect to the base edge, optionally recapitulating anisotropy in native heart valves.

17. The method of claim 1, wherein the target is non-planar.

18-21. (canceled)

22. The method of claim 17, wherein the target comprises a pattern of ridges and valleys configured to produce a fiber matrix in the size and shape of a heart valve leaflet having a base edge and an apex, and wherein the ridges and valleys are arranged to produce a convex arrangement of fibers with respect to the base edge, optionally recapitulating anisotropy in native heart valves.

23. The method of claim 22, wherein the target is shaped to produce a fiber matrix in the shape of a heart valve.

24. (canceled)

25. (canceled)

26. The method of claim 1, wherein the target comprises a siloxane composition and a repeated three-dimensional pattern or tessellation of raised shapes with a periodicity of less than 500 μm and the siloxane composition comprises a conductor or the target further comprises a conductive layer deposited over at least a portion of the surface of the target.

27-31. (canceled)

32. The method of claim 1, wherein the target comprises a siloxane composition and a repeated three-dimensional pattern or tessellation of raised shapes with a periodicity of less than 500 μm and the shapes of the repeated three-dimensional pattern or tessellation are not square or rectangular.

33-35. (canceled)

36. The method of claim 1, wherein the polymer fibers are prepared from a bioerodable polymer.

37. (canceled)

38. (canceled)

39. A system for the production of variably anisotropic fiber matrices, comprising:
an electrodeposition target attached to an electrical voltage source comprising:
an electrodeposition surface comprising a non-homogenous pattern of alternating ridges and valleys extending generally perpendicularly to a fiber alignment direction to produce an aligned electrical field for depositing the polymer fibers in the fiber alignment direction, optionally wherein the periodicity, of the ridges or raised shapes is 500 μm or less; or
a siloxane composition having an electrodeposition surface comprising a repeated three-dimensional pattern or tessellation of raised shapes with a periodicity of less than 500 μm;
an electrodeposition nozzle attached to an electrical voltage source; and
a polymer solution reservoir and pump configured to supply a polymer solution to the electrodeposition nozzle.

40. A method of making a vascular graft or heart valve graft, comprising:
seeding a polymer matrix structure prepared according to claim 1, shaped as a vascular graft or heart valve, with endothelial cells, vascular smooth muscle cells, or precursors of endothelial cells or vascular smooth muscle cells;
if precursors of endothelial cells or vascular smooth muscle cells are seeded, culturing the polymer matrix structure under conditions for differentiating the precursor cells into endothelial cells or vascular smooth muscle cells; and
culturing the cells to expand the number of cells on the polymer matrix structure.

41. A method of making a graft for tissue replacement or repair in a patient, comprising:
seeding a polymer matrix structure prepared according to claim 1, shaped as a graft for the tissue to be repaired or replaced, with cells or precursors of the cells;
if precursors of the cells are seeded, culturing the polymer matrix structure under conditions for differentiating the precursor cells into cells of the tissue to be repaired or replaced; and
culturing the cells to expand the number of cells on the polymer matrix structure.

42. A method of repairing or replacing tissue, such as vascular or heart valve tissue, in a patient, comprising implanting in the patient a polymer matrix structure or graft prepared according to claim 1.

43-46. (canceled)
